(19) United States
(12) Patent Application Publication
(10) Pub. No.: US 2023/0408522 A1
(43) Pub. Date: Dec. 21, 2023

(54) MULTI-PROTEIN CLASSIFIER FOR DETECTING EARLY-STAGE OVARIAN CANCER

(71) Applicant: REGENTS OF THE UNIVERSITY OF MINNESOTA, Minneapolis, MN (US)

(72) Inventors: Amy Patrice Skubitz, Edina, MN (US); Kristin L.M. Boylan, St. Paul, MN (US); Ashley Jenna Petersen, Minneapolis, MN (US); Timothy Kaehler Starr, Minneapolis, MN (US)

(21) Appl. No.: 18/207,970

(22) Filed: Jun. 9, 2023

Related U.S. Application Data
(60) Provisional application No. 63/350,953, filed on Jun. 10, 2022.

(51) Int. Cl. G01N 33/574; G16H 50/30

(52) U.S. Cl. CPC G01N 33/57449; G16H 50/30; G01N 2333/70546; G01N 2333/4725; G01N 2800/50

(57) ABSTRACT

In one aspect, the present disclosure relates to a method for determining risk of ovarian cancer in a patient, the method including: providing a biological sample from the patient; measuring a level of CA1225 in the biological sample; measuring a level of SEZ6L in the biological sample; and identifying that the patient is at risk of ovarian cancer based on the level of CA125 and the level of SEZ6L. In one or more embodiments, the method further includes measuring a level of HE4 and ITGAV. In another aspect, the present disclosure relates to a kit, system, or computer program for performing a method described herein.

### CROSS-REFERENCE TO RELATED APPLICATION

This application claims the benefit of U.S. Provisional Patent Application No. 63/350,953, filed Jun. 10, 2022, which is incorporated herein by reference in its entirety

### SUMMARY

This disclosure describes, in one aspect, a method for determining risk of ovarian cancer in a patient, the method including: providing a biological sample from the patient; measuring a level of mucin 16 (CA125) in the biological sample; measuring a level of seizure 6-like protein (SEZ6L) in the biological sample; and identifying that the patient is at risk of ovarian cancer based on the level of CA125 and the level of SEZ6L. In one or more embodiments, wherein identifying includes identifying a normal level of CA125, comparing the level of CA125 measured in the biological sample to the normal level of CA125, identifying that the level of CA125 measured in the biological sample is greater than the normal level of CA125, identifying a normal level of SEZ6L, comparing the level of SEZ6L measured in the biological sample to the normal level of SEZ6L, and identifying that the level of SEZ6L measured in the biological sample is less than the normal level of SEZ6L.

In one or more embodiments, a method further includes measuring a level of human epididymis protein 4 (HE4) in the biological sample, measuring a level of integrin alpha-V (ITGAV) in the biological sample, and identifying that the patient is at risk of ovarian cancer based on the levels of CA125, SEZ6L, HE4, and ITGAV. In one or more of these embodiments, identifying includes identifying a normal level of HE4, comparing the level of HE4 measured in the biological sample to the normal level of HE4, identifying that the level of HE4 measured in the biological sample is greater than the normal level of HE4, identifying a normal level of ITGAV, comparing the level of ITGAV measured in the biological sample to the normal level of ITGAV, and identifying that the level of ITGAV measured in the biological sample is less than the normal level of ITGAV.

In one or more embodiments, the biological sample includes serum, whole blood, plasma, saliva, urine, mucus, ascites fluid, a cervical swab, a vaginal swab, fine needle aspirate, and/or biopsied cells.

In another aspect, the present disclosure relates to a method for determining an ovarian cancer risk score in a patient including providing a biological sample, measuring a level of CA125 protein, a level of HE4 protein, a level of ITGAV protein, and a level of SEZ6L protein in the biological sample, providing levels of CA125 protein, REA protein, ITGAV protein, and SEZ6L, protein from at least one reference sample, determining, for each protein, a difference in the level of protein in the biological sample and the reference sample, thereby providing a normalized level of each protein, weighting the normalized level of CA125 using a first coefficient a, wherein a is a positive value, weighting the normalized level of HE4 using a second coefficient b, wherein b is a positive value, weighting the normalized level of ITGAV using a third coefficient c, wherein c is a negative value, weighting the normalized level of SEZ6L using a fourth coefficient d, wherein d is a negative value, determining an intercept value X, and determining an ovarian cancer risk score for the patient, wherein the ovarian cancer risk score is calculated using Formula II:

$$Risk score \% = 100 * ( \frac{{e}^{X + a ( CA 125 ) + b ( HE 4 ) + c ( ITGAV ) + d ( SEZ 6 L )}}{1 + {e}^{X + a ( CA 125 ) + b ( HE 4 ) + c ( ITGAV ) + d ( SEZ 6 L )}} ) \qquad Formula II$$

wherein the ovarian cancer risk score indicates a percent chance the patient has ovarian cancer.

In one or more embodiments, a is a value from 0 to 2, b is a value from 0 to 2, c is a value from −2 to 0, d is a value from −2 to 0, and X is a value from −5 to 5. In one or more embodiments, the ovarian cancer risk score is calculated using Formula I:

$$Risk score \% = 100 * ( \frac{{e}^{- 3.43 + 0.959 ( CA 125 ) + 0.38 ( HE 4 ) + ( - 0.946 ) ( ITGAV ) + ( - 0.964 ) ( SEZ 6 L )}}{1 + {e}^{- 3.43 + 0.959 ( CA 125 ) + 0.38 ( HE 4 ) + ( - 0.946 ) ( ITGAV ) + ( - 0.964 ) ( SEZ 6 L )}} ) . \qquad Formula I$$

In one or more embodiments, the ovarian cancer is early-stage ovarian cancer. In one or more certain embodiments, ovarian cancer includes clear cell ovarian cancer, mucinous ovarian cancer, or endometroid ovarian cancer. In one or more embodiments, a method includes treating the patient for ovarian cancer.

In one or more embodiments, a method demonstrates higher sensitivity at a set specificity as compared to a method wherein only CA125 is analyzed.

In one or more embodiments, a method further includes providing a protein level of one or more of SCF (UniProt ID No.: P21583), FASLG (UniProt ID No.: P48023), XPNPEP2 (UniProt ID No.: O43895), TCL1A (UniProt ID No.: P56279), VEGFR-2 (UniProt ID No.: P35968), CEACAM1 (UniProt ID No.: P13688), TLR3 (UniProt ID No.: O15455), CYR61 (UniProt ID No.: O00622), GPNMB (UniProt ID No.: Q14956), CPE (UniProt ID No.: P16870), LY9 (UniProt ID No.: Q9HBG7), ERBB2 (UniProt ID No.: P04626), GPC1 (UniProt ID No.: P35052), IFN-γ-R1 (UniProt ID No.: P15260), CD48 (UniProt ID No.: P09326), RET (UniProt ID No.: P07949), ICOSLG (UniProt ID No.: O75144), CTSV (UniProt ID No.: O60911), and MIA (UniProt ID No.: Q16674); and identifying a patient with below normal levels of the one or more proteins as at risk for ovarian cancer.

In one or more embodiments, a method further includes providing a protein level of one or more of MK (UniProt ID No.: P21741), IL6 (UniProt ID No.: P05231), ESM-1 (UniProt ID No.: Q9NQ30), hK11 (UniProt ID No.: Q9UBX7), ADAM-TS 15 (UniProt ID No.: Q8TE58), SYND1 (UniProt ID No.: P18827), CXCL13 (UniProt ID No.: O43927), TFPI-2 (UniProt ID No.: P48307), FR-α (UniProt ID No.: P15328), KLK13 (UniProt ID No.: Q9UKR3), MSLN (UniProt ID No.: Q13421), NECT4 (UniProt ID No.: Q96NY8), TNFRSF6B (UniProt ID No.: O95407), FCRLB (UniProt ID No.: Q6BAA4), and AREG (UniProt ID No.: P15514); and identifying a patient with above normal levels of the one or more proteins as at risk for ovarian cancer.

In another aspect, the present disclosure relates to a kit including a first reagent to measure a level of CA125 protein in a biological sample, the first reagent including an antibody and an oligonucleotide; a second reagent to measure a level of HE4 protein in a biological sample, the second reagent including an antibody and an oligonucleotide; a third reagent to measure a level of ITGAV protein in a biological sample, the third reagent including an antibody and an oligonucleotide; a fourth reagent to measure a level of SEZ6L protein in a biological sample, the fourth reagent including an antibody and an oligonucleotide; and a plate including wells, wherein a first well includes the first reagent, a second well includes the second reagent, a third well includes the third reagent, and a fourth well includes the fourth reagent. In one or more embodiments, each of the first, second, third, and fourth reagents includes an antibody and an oligonucleotide.

In another aspect, the present disclosure relates to a system for performing a method described herein.

In another aspect, the present disclosure relates to a computer program for performing a method described herein. In one or more embodiments, a computer program includes a non-transitory computer readable medium on which is provided program instructions for steps of providing levels of CA125 protein, HE4 protein, ITGAV protein, and SEZ6L protein from a biological sample from a patient; providing levels of CA125 protein, HE4 protein, ITGAV protein, and SEZ6L, protein from at least one reference sample; determining, for each protein, a difference in the level of protein in the biological sample and the reference sample, thereby providing a normalized level of each protein; weighting the normalized level of CA125 using a first coefficient a, wherein a is a positive value; weighting the normalized level of HE4 using a second coefficient b, wherein b is a positive value; weighting the normalized level of ITGAV using a third coefficient c, wherein c is a negative value; weighting the normalized level of SEZ6L using a fourth coefficient d, wherein d is a negative value; determining an intercept value X; and determining an ovarian cancer risk score for the patient, wherein the ovarian cancer risk score is calculated using Formula II:

$$Risk score \% = 100 * ( \frac{{e}^{X + a ( CA 125 ) + b ( HE 4 ) + c ( ITGAV ) + d ( SEZ 6 L )}}{1 + {e}^{X + a ( CA 125 ) + b ( HE 4 ) + c ( ITGAV ) + d ( SEZ 6 L )}} ) \qquad Formula II$$

wherein the ovarian cancer risk score indicates a percent chance the patient has ovarian cancer.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Defining proteins that may be sensitive to preanalytical variation. (A) The standardized mean differences (SMDs; i.e., t-statistics) in protein levels between those with and without cancer were compared for Texas (TX) vs. Minnesota (MN). Blue points are proteins that were significantly differentially expressed between institutions (e.g., over-expressed in MN cancers vs. under-expressed in TX cancers). (B) The same plot as in (A), except only the proteins tested to determine their preanalytical variation in Shen et al. (Clin Chem Lab Med. 56(4):582-594, 2018) are plotted. (C) Shen et al.'s measurement of protein instability (sum of ΔNPX values; higher values indicate more instability) was compared to the p-values for differential effects by institution, where small p-values are evidence of instability. The differential effect by institution p-values for all differentially expressed proteins are in Table 5, along with the mean differences by institution.

FIG. 2. Hierarchical clustering of 452 serum samples from Cohort #1 based on 67 proteins (excluding FOLR3). Blue indicates proteins with high levels of expression, while yellow indicates proteins with low levels of expression. Three major clusters were identified. The three color bands at the bottom identify samples. Band #1, Sample type and institution: Light red, 70 early-stage ovarian cancer (TX); dark red, 46 early-stage ovarian cancer (MN); light green, 275 healthy (TX); dark green, 61 healthy (MN). Band #2, Overall sample type: 116 cancer (red) and 336 healthy (green). Band #3, ovarian cancer subtypes: 53 serous (red), 21 endometrioid (blue), 14 clear cell (yellow), 15 mucinous (green), and 13 mixed (brown).

FIG. 3. Development of a multi-protein classifier from samples in Cohort #1. (A) PROSEEK Oncology II (Olink Proteomics AB, Uppsala, Sweden) normalized protein expression (NPX) values were plotted with the median and 25^{th} and 75^{th} percentiles for the healthy patients (blue) and the 5 subtypes of early-stage ovarian cancer (pink) for the four proteins included in the multi-protein classifier. (B) ROC curves for the multi-protein classifier and each of the four individual proteins included in the multi-protein classifier.

FIG. 4. Validation of the multi-protein classifier for use with Cohort #2, a second cohort of early-stage ovarian cancer samples. (A) PROSEEK NPX values of the proteins included in the early-stage multi-protein classifier between healthy controls and early-stage ovarian cancer samples from Cohort #2. The median and 25^{th} and 75^{th} percentiles are shown in all plots. (B) Receiver operating characteristics (ROC) curves for the early-stage multi-protein classifier applied to the Cohort #2 samples and individual proteins included in the early-stage multi-protein classifier.

FIG. 5. Validation of the multi-protein classifier with serum samples from women with benign ovarian conditions. (A) Comparison of the NPX values for the proteins included in the multi-protein classifier between healthy controls, benign samples, and the early-stage ovarian cancer samples from both cohorts of samples. (B) Predicted cancer risk scores stratified by true cancer status for the early-stage ovarian cancer, benign, and healthy control samples from Cohort #1 and Cohort #2. The median and 25^{th} and 75^{th} percentiles are shown in all plots.

FIG. 6. Validation of the multi-protein classifier with serum samples from women with late-stage ovarian cancer. (A) Predicted cancer risk scores with median and 25^{th} and 75^{th} percentiles stratified by true cancer status for late-stage ovarian cancer samples (n=61 ovarian cancer samples; n=88 healthy controls) from Skubitz et al. (Cancer Pre Res (Phila). 2019; 12(3):171-184). (B) ROC curve for the early-stage multi-protein classifier applied to the late-stage samples.

FIG. 7. Plots of 452 serum samples clustered by 67 proteins. (A) PCA plot showing 116 early-stage ovarian cancer samples (red) separate from the 336 healthy control samples (green) in Cohort #1. (B) t-distributed stochastic neighbor embedding (t-SNE) plot showing 116 early-stage ovarian cancer samples (red; clustered in the upper left corner of the plot) separate from the 336 healthy control samples (green) in Cohort #1. I t-SNE plot; CA125 ELISA values>35 (5.13 log 2)=red. (D) t-SNE plot; HE4>8.8=red. (E) t-SNE plot; ITGAV<2.6=red. (F) t-SNE plot; SEZ6L<2.1=red.

FIG. 8. Normalization of Cohort #2 using “bridge” samples from Cohort #1. (A) Prior to normalization, the association between the NPX values from Cohort #1 vs. Cohort #2 for the 22 bridge samples across all 68 proteins. (B) The protein-specific normalization factors for the 68 proteins. (C) Protein fold changes in Cohort #1 vs. Cohort #2.

FIG. 9. Tuning to determine a limited set of proteins to include in the multi-protein classifier. A multi-protein classifier was developed to differentiate healthy controls from early-stage ovarian cancer cases using least absolute shrinkage and selection operator (LASSO) logistic regression with the tuning parameter chosen using 10-fold cross-validation to be that with cross-validation error within 1 standard error of the minimum cross-validation error (“lambda.1se”). Summaries of the classification accuracy were estimated using the predicted probabilities from the held-out cross-validation folds. To obtain confidence intervals (Cis) for area under the curve (AUC), ROC (0.05), and ROC (0.02) for the multi-protein classifier, the bias-corrected bootstrap case cross-validation method was used. The difference in AUCs between different classifiers was tested using a bootstrap method for correlated ROC curves.

### DETAILED DESCRIPTION OF ILLUSTRATIVE EMBODIMENTS

This disclosure describes a method for determining risk of ovarian cancer in a patient. Generally, the method includes measuring serum levels of mucin 16 (CA125, also referred to in the art as CA 125 or CA-125), human epididymis protein 4 (HE4), integrin alpha-V (ITGAV), and seizure 6-like protein (SEZ6L). A patient with above normal levels of CA125 and HE4 and below normal levels of ITGAV and SEZ6L has an increased risk of ovarian cancer. The method can identify early-stage ovarian cancer in patients who may not yet show symptoms or clinical signs of ovarian cancer.

Ovarian cancer is a leading cause of cancer deaths in women in the United States. Due to vague symptoms and lack of adequate screening tests, most women are not diagnosed with ovarian cancer until it is advanced and the five-year survival rate is −30%. In contrast, for women diagnosed with stage I ovarian cancer, limited to the ovaries, the long-term survival rate is almost 90% and for stage II, limited to the pelvis, the survival rate is 70%, highlighting the need for strategies for earlier detection.

CA125, a well-known ovarian cancer biomarker, is not expressed in −20% of ovarian cancers and therefore is not adequately sensitive to screen the general population for ovarian cancer. It is possible that detecting biomarkers complementary to CA125 may increase the sensitivity of detecting early-stage disease. In addition to proteins, other molecules have been explored as potential biomarkers for ovarian cancer. Autoantibodies against cancer antigens such as TP53 have been identified in 20-30% of ovarian cancer cases tested, and may provide additional lead time over CA125. Serum microRNAs have been identified as candidate ovarian cancer biomarkers, and circulating tumor DNA has also been tested as a method for early detection.

New technology has been developed that makes it possible to measure levels of multiple protein biomarkers simultaneously in very small volumes of serum or plasma. The proximity extension assay (PEA, Olink Proteomics AB, Uppsala, Sweden) permits simultaneous quantification of 92 disease-related protein biomarkers, using sample volumes as low as 1 μl. PEA is an innovative technology that combines the specificity of antibody-based detection methods with the sensitivity of PCR, allowing multiplex biomarker quantification with high precision.

This disclosure describes measuring protein levels in sera collected at two different institutions from women diagnosed with early-stage ovarian cancer (all subtypes) using the PROSEEK Oncology II panel (Olink Proteomics AB, Uppsala, Sweden). Using these data, a multi-protein classifier was developed that could discriminate between early-stage ovarian cancer and healthy controls. This disclosure further describes using a second cohort of serum samples collected from women at four different institutions to validate the classifier and establish its predictive value using sera from women with late-stage ovarian cancer and benign ovarian conditions.

In one or more embodiments, a method includes providing a biological sample from a patient. While described in the context of an exemplary method using a serum sample, the methods of the present disclosure may be practiced with other biological samples. In one or more embodiments, a biological sample includes blood, plasma, serum, urine, ascites fluid, vaginal swabs, cervical swabs, mucus, or saliva. In one or more embodiments, a biological sample may include a biopsy sample, such as a solid tissue sample, a fine needle aspirate, or a liquid biopsy (most of those listed above are liquid samples). The methods described herein may be compatible with samples collected for detection of other gynecological cancers, such as cervical cancer screening samples or detection of human papilloma virus (e.g. cervical swabs or vaginal swabs). The methods described herein may be practiced with a sample that is fresh, cryopreserved, or chemically preserved.

### Cohort #1 Demographics

The PROSEEK Oncology II panel (Olink Proteomics AB, Uppsala, Sweden) was used to quantify expression of 92 cancer-related proteins in 1 μl of serum from 336 healthy women and 116 women with early-stage ovarian cancer from the University of Minnesota and MD Anderson Cancer Center (Cohort #1; Table 1). The ovarian cancer samples were comprised of the major epithelial subtypes of ovarian cancer, with almost half of the samples from women diagnosed with high grade serous ovarian cancer (HGSOC; 46%). The remaining ovarian cancer samples were from women with endometrioid (18%), mucinous (13%), clear cell (12%), or with mixed ovarian cancer subtypes (11%).

TABLE 1
Patient demographic information for Cohort #1 and Cohort #2

| | Cohort #1 (Discovery) | | Cohort #2 (Validation) | |
|---|---|---|---|---|
| | | Early stage | | Early-stage |
| | | ovarian | | ovarian |
| | Healthy | cancer | Healthy | cancer |
| | (n = 336) | (n = 116) | (n = 467) | (n = 192) |
| Location | | | | |
| MN | 61 (18%) | 46 (40%) | | |
| TX | 275 (82%) | 70 (60%) | | |
| Fox Chase | | | 226 (48%) | 55 (29%) |
| Italy—Milan | | | 144 (31%) | 86 (45%) |
| OHSU | | | 7 (1%) | 12 (6%) |
| BWH-Harvard | | | 90 (19%) | 39 (20%) |
| Age | | | | |
| Mean (SD) | 66.4 (7.6) | 58.5 (12.5) | 55.1 (11.5) | 56.3 (11.5) |
| Median | 67.0 | 58.5 | 54.0 | 56.0 |
| Range | 48-87 | 19-85 | 24-85 | 24-85 |
| CA125 value | | | | |
| Median (Q1, Q3) | 10.4 | 98.3 | ND | 143 |
| | (7.7, 14.3) | (42.9, 379) | | (42.3, 543) |
| Range | 0-69 | 7-22780 | ND | 2-12219 |
| Subtype | | | | |
| HGSOC | | 53 (46%) | | 76 (40%) |
| Endometrioid | | 21 (18%) | | 50 (26%) |
| Clear cell | | 14 (12%) | | 36 (19%) |
| Mucinous | | 15 (13%) | | 16 (8%) |
| Mixed | | 13 (11%) | | 11 (6%) |
| Other | | 0 (0%) | | 3 (2%) |
| Stage | | | | |
| I | | 73 (63%) | | 119 (62%) |
| II | | 43 (37%) | | 73 (38%) |

### Identification of Unstable Proteins

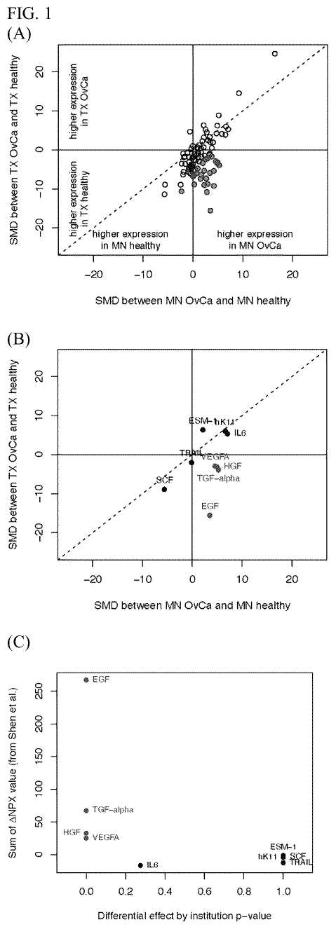

The PROSEEK assay (Olink Proteomics AB, Uppsala, Sweden) uses proximity extension assay (PEA) technology in which oligonucleotide-labeled antibody pairs are used to quantify proteins by real-time polymerase chain reaction (PCR). To determine whether any of the protein measurements may have been sensitive to preanalytical variation during sample collection or processing, the standardized mean differences (SMDs) in protein levels were compared between subjects with and without cancer for samples from MD Anderson (TX) and the University of Minnesota (MN) (FIG. 1A). Twenty-four proteins were significantly differentially expressed between institutions (e.g., over-expressed in MN cancer samples and under-expressed in TX cancer samples; FIG. 1A, blue circles). The SMDs in protein levels for the nine proteins in the study that were also examined in a previous study (Shen et al., Clin Chem Lab Med. 56(4):582-594, 2018) are shown in FIG. 1B. When the protein instability measurement from Shen et al. (sum of ΔNPX values, with higher values indicating more instability) was compared to the p-values for differential effects by institution in the present study (where small p-values are evidence of instability), all four of the proteins with significant p-values for differential effects by institution (blue filled circles in FIG. 1B) also had the highest sum of ΔNPX values (blue circles in FIG. 1C). These results identify proteins that are unstable and sensitive to preanalytical variation. Therefore, the 24 proteins that were significantly differentially expressed between TX and MN were removed from downstream analysis, due to concern that they would not serve well in the development of future clinical biomarker panels.

TABLE 5
The mean differences in protein levels between cancer and healthy samples by
institution for the 24 proteins with differential expression by institution for Cohort #1

| | | | | | | Differential |
|---|---|---|---|---|---|---|
| | | | | | | effect by |
| | UniProt | MN mean | MN | TX mean | TX | institution |
| Protein | ID | difference | SMD | difference | SMD | p-value |
| EGF | P01133 | 0.98 | 3.51 | −2.97 | −15.58 | <0.001 |
| VIM | P08670 | 0.57 | 4.21 | −0.85 | −9.25 | <0.001 |
| SPARC | P09486 | 0.13 | 2.81 | −0.34 | −10.82 | <0.001 |
| TXLNA | P40222 | 0.42 | 2.91 | −0.86 | −8.78 | <0.001 |
| SCAMP3 | O14828 | 0.53 | 2.29 | −1.44 | −9.04 | <0.001 |
| TGF-alpha | P01135 | 0.71 | 5.27 | −0.36 | −3.91 | <0.001 |
| LYN | P07948 | 0.27 | 4.18 | −0.24 | −5.41 | <0.001 |
| ABL1 | P00519 | 0.35 | 2.65 | −0.66 | −7.35 | <0.001 |
| PPY | P01298 | 0.88 | 3.41 | −1.05 | −5.93 | <0.001 |
| S100A11 | P31949 | 0.56 | 4.93 | −0.27 | −3.43 | <0.001 |
| HGF | P14210 | 0.55 | 4.97 | −0.24 | −3.22 | <0.001 |
| MetAP 2 | P50579 | 0.20 | 1.56 | −0.65 | −7.44 | <0.001 |
| VEGFA | P15692 | 0.58 | 4.59 | −0.26 | −2.97 | <0.001 |
| S100A4 | P26447 | 0.03 | 0.25 | −0.60 | −8.87 | <0.001 |
| FADD | Q13158 | 0.22 | 1.69 | −0.56 | −6.40 | <0.001 |
| CDKNIA | P38936 | 0.30 | 2.27 | −0.49 | −5.35 | <0.001 |
| ITGB5 | P18084 | −0.15 | −2.30 | −0.47 | −10.63 | 0.003 |
| ANXA1 | P04083 | 0.74 | 3.79 | −0.23 | −1.72 | 0.004 |
| GZMB | P10144 | 0.10 | 0.69 | −0.60 | −6.04 | 0.006 |
| MAD | Q99717 | 0.00 | 0.03 | −0.29 | −6.80 | 0.009 |
| homolog 5 | | | | | | |
| ERBB3 | P21860 | 0.01 | 0.14 | −0.21 | −6.39 | 0.02 |
| ADAM 8 | P78325 | 0.27 | 2.93 | −0.13 | −2.16 | 0.02 |
| VEGFR-3 | P35916 | 0.05 | 0.95 | −0.17 | −4.80 | 0.04 |
| CEACAM5 | P06731 | 0.38 | 3.34 | −0.10 | −1.26 | 0.04 |

The mean differences in protein levels between cancer and healthy samples by institution, as well as the standardized mean differences (SMDs; i.e., t-statistics) are shown for Cohort #1. Negative values indicate under-expression in the cancer samples (vs. healthy) for that institution, while positive values indicate over-expression. The proteins are shown for which there was evidence of a differential effect by institution (Holm′s adjusted p < 0.05).

### Identification of Candidate Biomarkers for Early Stage Ovarian Cancer

PROSEEK Oncology II assay measurements for both CA125 and HE4 correlate with the clinical values or enzyme linked immunosorbent assay (ELISA) measurements in serum samples obtained from late-stage high grade serous ovarian cancer. In the present study, a similar comparison was made comparing the PROSEEK NPX values with the clinical values or ELISA measurements for CA125. Again, the measurements were highly correlated (r=0.83).

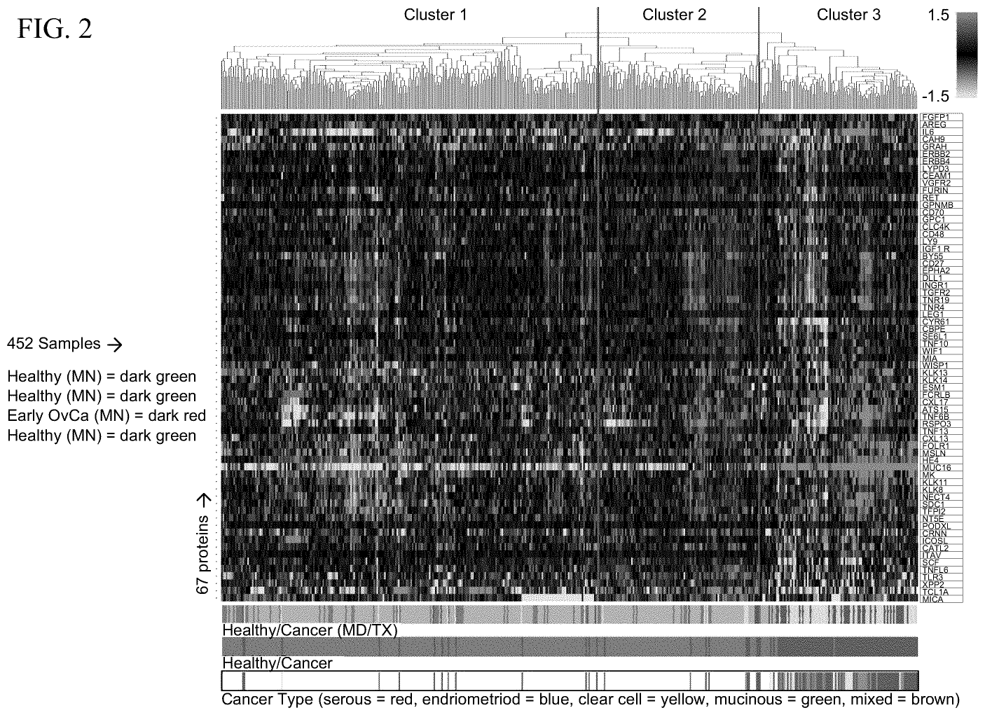

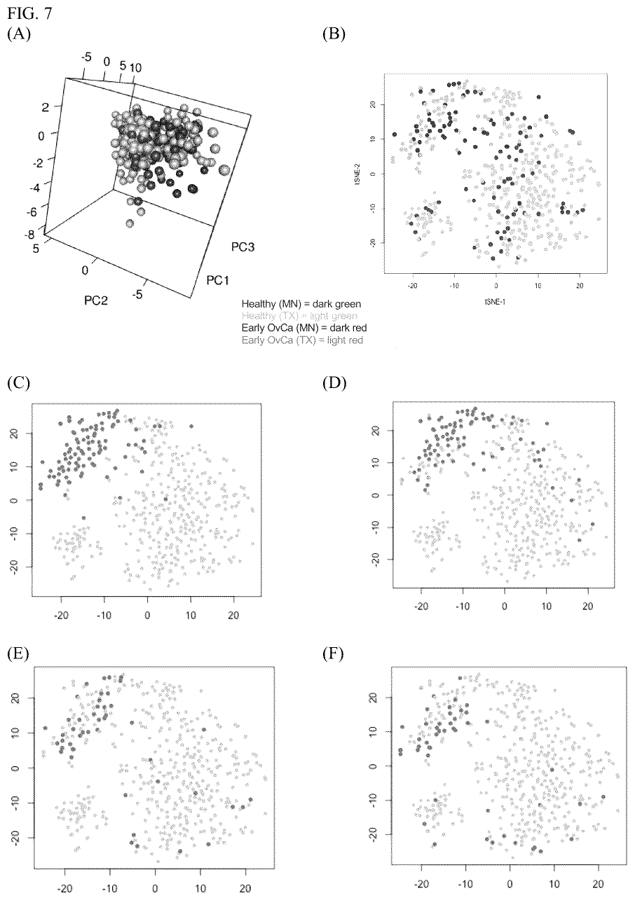

Unsupervised clustering of the 452 samples was also performed in Cohort #1 based on 67 proteins to visualize the protein expression differences between the samples from the two institutions. Plotting of the first three principal components (FIG. 7A) and t-SNE plots (FIG. 7B) show that the early-stage ovarian cancer serum samples separate from the healthy serum samples regardless of institution of origin. Similar to the PCA and t-SNE analysis, unsupervised hierarchical clustering identified major clusters of cancer vs. healthy (FIG. 2). Clusters 1 and 2 are composed primarily of samples from healthy controls, while cluster 3 is primarily from ovarian cancer samples, with some exceptions scattered throughout. The hierarchical clustering revealed a group of proteins that are expressed at elevated levels in ovarian cancer serum samples including: CA125 (MUC16), HE4 (WFDC2), MSLN, MK, KLK8, KLK11, NECT4, FOLR1, KLK13, KLK14, and IL6.

The two clusters formed by samples from healthy individuals are divided by a general upregulation vs. downregulation of all proteins. There is no evidence of batch effect between the sources, as samples from both institutions are interspersed.

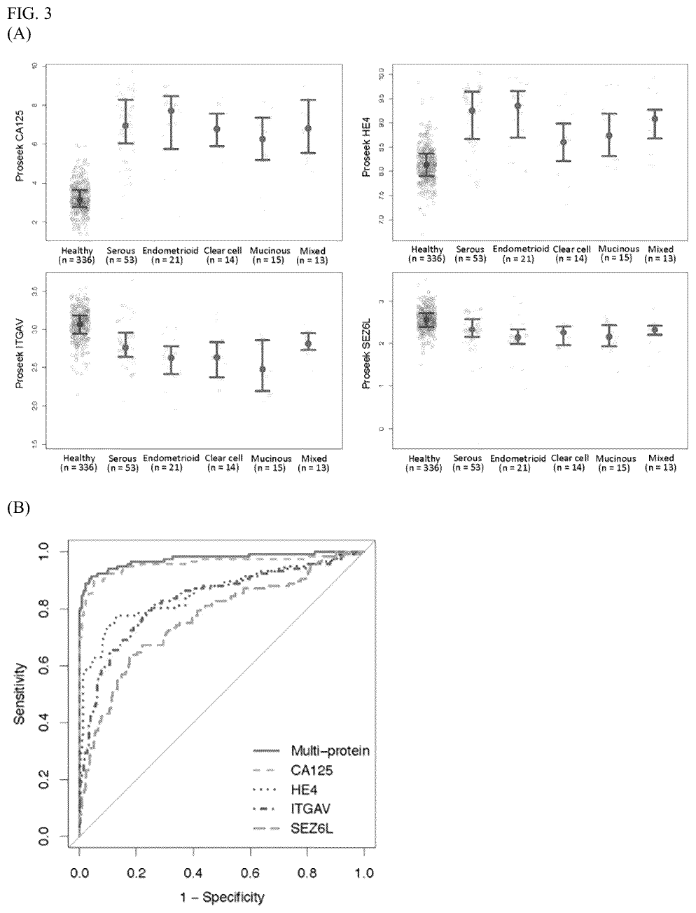

By FDR-adjusted two-sample t-tests, mean levels of 38 of the 68 proteins differed significantly (p<0.05) between the early stage (I-II) ovarian cancer and healthy samples (Table 2). 17 of these proteins were elevated in the ovarian cancer samples compared to the healthy control samples, including CA125 and HE4. The PROSEEK NPX values for CA125 and HE4 were elevated in ovarian cancer samples from all subtypes (FIG. 3A). The mean NPX expression values by diagnosis of all 68 proteins is provided in Table 6.

TABLE 2
Mean (standard deviation) of NPX values for the
38 proteins in Cohort #1 significantly different
between ovarian cancer and healthy controls.

| | | | Early stage | |
|---|---|---|---|---|
| | | Healthy | ovarian cancer | |
| Protein | UniProt ID | (n = 336) | (n = 116) | p-value |
| CA125 | Q8WXI7 | 3.21 (0.77) | 6.73 (1.68) | <0.001 |
| HE4 | Q14508 | 8.14 (0.39) | 9.02 (0.65) | <0.001 |
| ITGAV | P06756 | 3.05 (0.21) | 2.70 (0.32) | <0.001 |
| MK | P21741 | 6.40 (0.60) | 7.17 (0.95) | <0.001 |
| SCF | P21583 | 8.89 (0.41) | 8.22 (0.85) | <0.001 |
| IL6 | P05231 | 2.89 (1.26) | 4.21 (1.68) | <0.001 |
| SEZ6L | Q9BYH1 | 2.56 (0.27) | 2.22 (0.44) | <0.001 |
| FASLG | P48023 | 8.97 (0.48) | 8.58 (0.52) | <0.001 |
| ESM-1 | Q9NQ30 | 8.98 (0.57) | 9.44 (0.62) | <0.001 |
| hK11 | Q9UBX7 | 6.18 (0.44) | 6.74 (0.86) | <0.001 |
| ADAM-TS 15 | Q8TE58 | 1.86 (0.63) | 2.41 (0.90) | <0.001 |
| XPNPEP2 | O43895 | 8.06 (0.58) | 7.61 (0.73) | <0.001 |
| SYND1 | P18827 | 6.06 (0.50) | 6.51 (0.82) | <0.001 |
| CXCL13 | O43927 | 7.66 (0.61) | 8.14 (0.86) | <0.001 |
| TFPI-2 | P48307 | 7.57 (0.49) | 7.98 (0.76) | <0.001 |
| TCL1A | P56279 | 4.01 (1.22) | 3.28 (1.31) | <0.001 |
| FR-α | P15328 | 6.57 (0.48) | 7.12 (1.08) | <0.001 |
| KLK13 | Q9UKR3 | 3.41 (0.75) | 3.84 (0.87) | <0.001 |
| VEGFR-2 | P35968 | 6.70 (0.28) | 6.56 (0.30) | <0.001 |
| CEACAM1 | P13688 | 6.02 (0.24) | 5.91 (0.25) | <0.001 |
| TLR3 | O15455 | 4.93 (0.67) | 4.56 (0.87) | <0.001 |
| MSLN | Q13421 | 3.12 (0.66) | 3.55 (1.03) | <0.001 |
| CYR61 | O00622 | 5.70 (0.49) | 5.37 (0.83) | 0.001 |
| GPNMB | Q14956 | 6.07 (0.19) | 5.97 (0.24) | 0.001 |
| CPE | P16870 | 3.95 (0.42) | 3.72 (0.58) | 0.002 |
| LY9 | Q9HBG7 | 5.17 (0.41) | 4.96 (0.53) | 0.003 |
| NECT4 | Q96NY8 | 4.03 (0.47) | 4.36 (0.92) | 0.004 |
| ERBB2 | P04626 | 7.44 (0.31) | 7.27 (0.49) | 0.004 |
| TNFRSF6B | O95407 | 5.10 (0.78) | 5.51 (1.13) | 0.005 |
| FCRLB | Q6BAA4 | 0.92 (0.52) | 1.18 (0.74) | 0.01 |
| GPC1 | P35052 | 4.64 (0.39) | 4.44 (0.55) | 0.01 |
| IFN-γ-R1 | P15260 | 4.68 (0.32) | 4.53 (0.43) | 0.01 |
| CD48 | P09326 | 5.86 (0.32) | 5.73 (0.42) | 0.01 |
| RET | P07949 | 5.35 (0.48) | 5.12 (0.67) | 0.01 |
| ICOSLG | O75144 | 5.94 (0.57) | 5.71 (0.73) | 0.03 |
| CTSV | O60911 | 3.74 (0.48) | 3.54 (0.64) | 0.03 |
| AREG | P15514 | 1.87 (0.57) | 2.07 (0.62) | 0.03 |
| MIA | Q16674 | 9.66 (0.29) | 9.55 (0.37) | 0.03 |

TABLE 6

| | | Healthy | OvCa | |
|---|---|---|---|---|
| Protein | UniProt ID | (n = 336) | (n = 116) | p-value |
| CA125 | Q8WXI7 | 3.21 (0.77) | 6.73 (1.68) | <0.001 |
| HE4 | Q14508 | 8.14 (0.39) | 9.02 (0.65) | <0.001 |
| ITGAV | P06756 | 3.05 (0.21) | 2.70 (0.32) | <0.001 |
| MK | P21741 | 6.40 (0.60) | 7.17 (0.95) | <0.001 |
| SCF | P21583 | 8.89 (0.41) | 8.22 (0.85) | <0.001 |
| IL6 | P05231 | 2.89 (1.26) | 4.21 (1.68) | <0.001 |
| SEZ6L | Q9BYH1 | 2.56 (0.27) | 2.22 (0.44) | <0.001 |
| FASLG | P48023 | 8.97 (0.48) | 8.58 (0.52) | <0.001 |
| ESM-1 | Q9NQ30 | 8.98 (0.57) | 9.44 (0.62) | <0.001 |
| hK11 | Q9UBX7 | 6.18 (0.44) | 6.74 (0.86) | <0.001 |
| ADAM-TS 15 | Q8TE58 | 1.86 (0.63) | 2.41 (0.90) | <0.001 |
| XPNPEP2 | O43895 | 8.06 (0.58) | 7.61 (0.73) | <0.001 |
| SYND1 | P18827 | 6.06 (0.50) | 6.51 (0.82) | <0.001 |
| CXCL13 | O43927 | 7.66 (0.61) | 8.14 (0.86) | <0.001 |
| TFPI-2 | P48307 | 7.57 (0.49) | 7.98 (0.76) | <0.001 |
| TCL1A | P56279 | 4.01 (1.22) | 3.28 (1.31) | <0.001 |
| FR-alpha | P15328 | 6.57 (0.48) | 7.12 (1.08) | <0.001 |
| KLK13 | Q9UKR3 | 3.41 (0.75) | 3.84 (0.87) | <0.001 |
| VEGFR-2 | P35968 | 6.70 (0.28) | 6.56 (0.30) | <0.001 |
| CEACAM1 | P13688 | 6.02 (0.24) | 5.91 (0.25) | <0.001 |
| TLR3 | O15455 | 4.93 (0.67) | 4.56 (0.87) | <0.001 |
| MSLN | Q13421 | 3.12 (0.66) | 3.55 (1.03) | <0.001 |
| CYR61 | O00622 | 5.70 (0.49) | 5.37 (0.83) | 0.001 |
| GPNMB | Q14956 | 6.07 (0.19) | 5.97 (0.24) | 0.001 |
| CPE | P16870 | 3.95 (0.42) | 3.72 (0.58) | 0.002 |
| LY9 | Q9HBG7 | 5.17 (0.41) | 4.96 (0.53) | 0.003 |
| NECT4 | Q96NY8 | 4.03 (0.47) | 4.36 (0.92) | 0.004 |
| ERBB2 | P04626 | 7.44 (0.31) | 7.27 (0.49) | 0.004 |
| TNFRSF6B | O95407 | 5.10 (0.78) | 5.51 (1.13) | 0.005 |
| FCRLB | Q6BAA4 | 0.92 (0.52) | 1.18 (0.74) | 0.01 |
| GPC1 | P35052 | 4.64 (0.39) | 4.44 (0.55) | 0.01 |
| IFN-gamma-R1 | P15260 | 4.68 (0.32) | 4.53 (0.43) | 0.01 |
| CD48 | P09326 | 5.86 (0.32) | 5.73 (0.42) | 0.01 |
| RET | P07949 | 5.35 (0.48) | 5.12 (0.67) | 0.01 |
| ICOSLG | O75144 | 5.94 (0.57) | 5.71 (0.73) | 0.03 |
| CTSV | O60911 | 3.74 (0.48) | 3.54 (0.64) | 0.03 |
| AREG | P15514 | 1.87 (0.57) | 2.07 (0.62) | 0.03 |
| MIA | Q16674 | 9.66 (0.29) | 9.55 (0.37) | 0.03 |
| CD27 | P26842 | 8.89 (0.44) | 9.05 (0.58) | 0.07 |
| TGFR-2 | P37173 | 6.11 (0.38) | 5.98 (0.53) | 0.10 |
| PODXL | O00592 | 2.84 (0.21) | 2.94 (0.41) | 0.11 |
| GZMH | P20718 | 3.28 (0.85) | 3.07 (0.81) | 0.12 |
| LYPD3 | O95274 | 3.70 (0.40) | 3.58 (0.53) | 0.16 |
| CRNN | Q9UBG3 | 4.81 (0.75) | 4.61 (0.82) | 0.16 |
| hK14 | Q9POG3 | 6.75 (0.57) | 6.98 (1.03) | 0.17 |
| IGFIR | P08069 | 3.19 (0.31) | 3.11 (0.35) | 0.28 |
| CD207 | Q9UJ71 | 3.14 (0.43) | 3.03 (0.55) | 0.46 |
| hK8 | O60259 | 6.95 (0.45) | 7.07 (0.68) | 0.48 |
| RSPO3 | Q9BXY4 | 3.16 (0.82) | 3.34 (0.99) | 0.58 |
| CD70 | P32970 | 3.21 (0.58) | 3.11 (0.58) | 0.84 |
| TNFSF13 | O75888 | 8.16 (0.39) | 8.09 (0.62) | 1 |
| TRAIL | P50591 | 7.69 (0.34) | 7.61 (0.55) | 1 |
| FURIN | P09958 | 9.12 (0.47) | 9.18 (0.61) | 1 |
| EPHA2 | P29317 | 2.66 (0.37) | 2.66 (0.55) | 1 |
| Gal-1 | P09382 | 6.74 (0.25) | 6.71 (0.31) | 1 |
| CAIX | Q16790 | 3.50 (0.63) | 3.44 (0.97) | 1 |
| ERBB4 | Q15303 | 5.15 (0.28) | 5.08 (0.61) | 1 |
| 5′-NT | P21589 | 10.2 (0.53) | 10.1 (0.69) | 1 |
| DLL1 | O00548 | 9.59 (0.37) | 9.59 (0.52) | 1 |
| FGF-BP1 | Q14512 | 5.13 (0.47) | 5.21 (0.67) | 1 |
| TNFRSF19 | Q9NS68 | 3.94 (0.52) | 3.88 (0.85) | 1 |
| CD160 | O95971 | 5.19 (0.57) | 5.18 (0.79) | 1 |
| TNFRSF4 | P43489 | 3.01 (0.46) | 3.10 (0.64) | 1 |
| MIC-A/B | Q29983, | 3.66 (1.33) | 3.78 (1.19) | 1 |
| | Q29980 | | | |
| WISP-1 | O95388 | 6.10 (0.63) | 6.10 (0.71) | 1 |
| CXL17 | Q6UXB2 | 3.86 (0.56) | 3.75 (0.90) | 1 |
| FR-gamma | P41439 | 6.78 (2.02) | 6.85 (2.07) | 1 |
| WIF-1 | Q9Y5W5 | 5.05 (0.41) | 5.05 (0.63) | 1 |

While some proteins were found to be elevated in the ovarian cancer samples compared to the healthy control samples, some proteins were found to be decreased in the ovarian cancer samples compared to the healthy control samples. While many diagnostic assays test for elevated levels of one or more analytes, decreased levels of one or more analytes also may be used to identify risk of ovarian cancer in a patient.

To summarize the sensitivity (true positive rate; the probability that an ovarian cancer specimen will be correctly identified as cancer) and specificity (true negative rate; the probability that a healthy control sample will be correctly identified as healthy) of each protein individually across all classification thresholds, the AUC was calculated for each of the 68 proteins. In total, 11 individual proteins had an estimated AUC of >0.70 (Table 3).

In one or more embodiments, a method provides a specificity of at least 90%, such as at least 91%, at least 92%, at least 93%, at least 94%, at least 95%, at least 96%, at least 97%, at least 98%, or at least 99%. In one or more preferred embodiments, a method provides a specificity of 95% or 98%.

Sensitivity of a given method is related to a predetermined level of specificity. Increased specificity is indicative of a decreased rate of false positive diagnoses. Increased sensitivity is indicative of a decreased rate of false negative diagnoses. The relative levels of sensitivity and specificity for a certain method may be determined based on the accepted risk of a false negative diagnosis and a false positive diagnosis. Thus, for example, a method with 95% specificity may be preferred over a comparable method with 98% specificity because the method with 95% specificity may have a higher level of sensitivity than the comparable method with 98% specificity.

In one or more embodiments, a method of the present disclosure is more sensitive at a set specificity than a comparable method that analyzes CA125 alone. Expression of CA125 alone may be analyzed to inform whether a patient may have a risk of ovarian cancer. However, the level of sensitivity of such a method is often unacceptable. The methods described herein analyze expression levels of additional proteins identified herein as indicative of a patient's risk of ovarian cancer. In one or more embodiments, a method described herein demonstrates higher sensitivity at a set specificity (e.g., 95%, 98%) than a comparable method wherein only CA125 is analyzed. This may be true of methods wherein one, two, three, four, five, six, seven, eight, nine, ten, 11, 12, 13, 14, 15, 16, 17, 18, 19, or 20 proteins in addition to CA125 are analyzed.

Typically, analysis of additional proteins increases sensitivity of a method at a given specificity. However, at a certain point, analyzing additional proteins provides diminishing improvements to sensitivity and may decrease specificity. In one or more embodiments, a method analyzes at most 50, at most 40, at most 30, at most 25, at most 24, at most 23, at most 22, at most 21, at most 20, at most 19, at most 18, at most 17, at most 16, at most 15, at most 14, at most 13, at most 12, at most 11, at most ten, at most nine, at most eight, at most seven, at most six, at most five, at most four, at most three, or at most two proteins.

TABLE 3
AUC and sensitivities at 95% and 98% specificity for the 11 proteins with AUC > 0.70
comparing women with ovarian cancer to healthy women in Cohort #1. Summaries are also
given for the proteins when combined with CA125.

| | | | Single-Protein Sensitivity | | Single-Protein Sensitivity | |
|---|---|---|---|---|---|---|
| | Single-Protein AUC | | at 95% Specificity | | at 98% Specificity | |
| Protein | Estimate (95% CI) | Rank | Estimate (95% CI) | Rank | Estimate (95% CI) | Rank |
| CA125 | 0.958 (0.928, 0.982) | 1 | 0.879 (0.802, 0.940) | 1 | 0.810 (0.707, 0.897) | 1 |
| HE4 | 0.857 (0.808, 0.901) | 2 | 0.612 (0.526, 0.716) | 2 | 0.578 (0.302, 0.672) | 2 |
| ITGAV | 0.832 (0.783, 0.878) | 3 | 0.440 (0.302, 0.621) | 3 | 0.276 (0.164, 0.440) | 4 |
| SCF | 0.778 (0.728, 0.825) | 4 | 0.336 (0.233, 0.448) | 6 | 0.293 (0.172, 0.371) | 3 |
| SEZ6L | 0.764 (0.709, 0.816) | 5 | 0.310 (0.207, 0.448) | 9 | 0.164 (0.078, 0.310) | 14 |
| IL6 | 0.762 (0.708, 0.812) | 6 | 0.310 (0.129, 0.474) | 9 | 0.129 (0.000, 0.233) | 22 |
| MK | 0.745 (0.685, 0.802) | 7 | 0.405 (0.310, 0.509) | 4 | 0.250 (0.052, 0.431) | 6 |
| ESM-1 | 0.718 (0.661, 0.772) | 8 | 0.250 (0.129, 0.353) | 17 | 0.121 (0.060, 0.224) | 28 |
| hK11 | 0.713 (0.651, 0.770) | 9 | 0.353 (0.224, 0.466) | 5 | 0.216 (0.138, 0.336) | 9 |
| ADAM-TS 15 | 0.710 (0.649, 0.768) | 10 | 0.259 (0.181, 0.379) | 16 | 0.233 (0.129, 0.310) | 8 |
| FASLG | 0.705 (0.647, 0.761) | 11 | 0.284 (0.172, 0.414) | 12 | 0.181 (0.026, 0.267) | 12 |
| | | | Protein + CA125 Sensitivity | | Protein + CA125 Sensitivity | |
| | Protein + CA125 AUC | | at 95% Specificity | | at 98% Specificity | |
| Protein | Estimate (95% CI) | Rank | Estimate (95% CI) | Rank | Estimate (95% CI) | Rank |
| CA125 | — | — | — | — | — | — |
| HE4 | 0.966 (0.944, 0.983) | 8 | 0.853 (0.784, 0.914) | 55 | 0.784 (0.664, 0.888) | 55 |
| ITGAV | 0.967 (0.941, 0.987) | 7 | 0.914 (0.845, 0.957) | 1 | 0.862 (0.776, 0.931) | 2 |
| SCF | 0.958 (0.927, 0.982) | 46 | 0.879 (0.802, 0.940) | 36 | 0.810 (0.707, 0.897) | 33 |
| SEZ6L | 0.974 (0.950, 0.992) | 1 | 0.905 (0.845, 0.957) | 3 | 0.897 (0.836, 0.948) | 1 |
| IL6 | 0.963 (0.935, 0.983) | 15 | 0.836 (0.759, 0.914) | 61 | 0.793 (0.707, 0.862) | 51 |
| MK | 0.959 (0.931, 0.982) | 29 | 0.862 (0.776, 0.931) | 52 | 0.784 (0.698, 0.871) | 55 |
| ESM-1 | 0.960 (0.931, 0.983) | 24 | 0.862 (0.802, 0.940) | 52 | 0.802 (0.716, 0.897) | 45 |
| hK11 | 0.953 (0.923, 0.976) | 65 | 0.828 (0.750, 0.914) | 66 | 0.776 (0.672, 0.853) | 60 |
| ADAM-TS 15 | 0.961 (0.931, 0.984) | 21 | 0.888 (0.810, 0.940) | 22 | 0.793 (0.716, 0.905) | 51 |
| FASLG | 0.973 (0.954, 0.988) | 2 | 0.914 (0.853, 0.974) | 1 | 0.836 (0.733, 0.931) | 11 |

Seven of these 11 proteins were elevated in the early stage (I-II) ovarian cancer samples compared to control samples, while ITGAV, SCF, SEZ6L, and FASLG were decreased. CA125 had the highest AUC (0.958, 95% CI: 0.928-0.982) and HE4 was second with an AUC of 0.857 (95% CI: 0.808-0.901).

The sensitivity at two fixed levels of specificity (95% and 98%) is shown for the 11 proteins with AUC>0.70 in Table 3. At 95% specificity, CA125 had a sensitivity of 0.879 (95% CI: 0.802-0.940) and HE4 had a sensitivity of 0.612 (95% CI: 0.526-0.716). Similarly, at 98% specificity, CA125 ranked first, with a sensitivity of 0.810 (95% CI: 0.707-0897) and HE4 ranked second with a sensitivity of 0.578 (95% CI: 0.302-0.672).

However, when the AUCs for the individual proteins in combination with CA125 were considered, HE4 was outperformed by multiple other proteins. When the performance in combination with CA125 was considered, HE4 no longer ranked at the top. Instead, SEZ6L and ITGAV had the highest sensitivities at 98% specificity.

### Development of a Multi-Protein Classifier for Early Stage Ovarian Cancer

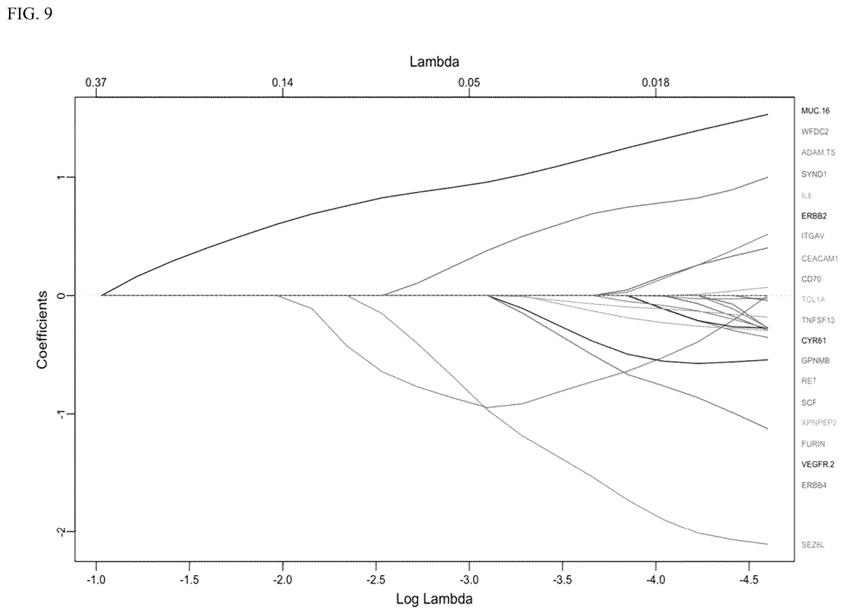

To improve the detection of ovarian cancer at an early stage over CA125 alone, a statistical learning method was used to develop a multi-protein classifier that could distinguish sera from early-stage ovarian cancer patients from that of healthy control women (FIG. 9). Using LASSO logistic regression to adaptively perform variable selection, the expression of 68 cancer-related proteins were considered as potential predictors with the optimal model combining the expression values of CA125 with three additional proteins (HE4, ITGAV, and SEZ6L) such that the predicted ovarian cancer risk score is equal to:

expit(−3.43+0.959×CA125+0.380×HE4+−0.946×ITGAV+−0.964×SEZ6L): Formula I

where expit(x)=e^{x}/(1+e^{x}).
Put into general terms, a risk score may be determined using Formula II:

$$Risk score \% = 100 * ( \frac{{e}^{- 3.43 + 0.959 ( CA 125 ) + 0.38 ( HE 4 ) + ( - 0.946 ) ( ITGAV ) + ( - 0.964 ) ( SEZ 6 L )}}{1 + {e}^{- 3.43 + 0.959 ( CA 125 ) + 0.38 ( HE 4 ) + ( - 0.946 ) ( ITGAV ) + ( - 0.964 ) ( SEZ 6 L )}} ) . \qquad Formula I$$

In one or more embodiments, a risk score may be calculated using Formula II, wherein X=−3.43, a=0.959, b=0.380, c=−0.946, and d=−0.964. The values of a, b, c, and d may be modified to normalize a sample to a reference sample.

In one or more embodiments, value “a” may be any value greater than zero. In one or more of these embodiments, value “a” may be at least 0.01, at least 0.05, at least 0.1, at least 0.15, at least 0.2, at least 0.25, at least 0.3, at least 0.35, at least 0.4, at least 0.5, at least 0.6, at least 0.7, at least 0.8, at least 0.9, at least 1.0, at least 1.1, at least 1.2, at least 1.4, at least, at least 1.6, at least 1.8, or at least 2.0. In one or more embodiments, value “a” may be at most 5, such as at most 4, at most 3, or at most 2. In one or more preferred embodiments, value “a” may be from 0 to 2, such as 0.959.

In one or more embodiments, value “b” may be any value greater than zero. In one or more of these embodiments, value “b” may be at least 0.01, at least 0.05, at least 0.1, at least at least 0.2, at least 0.25, at least 0.3, at least 0.35, at least 0.4, at least 0.5, at least 0.6, at least 0.7, at least 0.8, at least 0.9, at least 1.0, at least 1.1, at least 1.2, at least 1.4, at least 1.5, at least 1.6, at least 1.8, or at least 2.0. In one or more embodiments, value “b” may be at most 5, such as at most 4, at most 3, or at most 2. In one or more preferred embodiments, value “b” may be from 0 to 1, such as 0.380.

In one or more embodiments, value “c” may be any value less than zero. In one or more of these embodiments, value “c” may be at most −0.01, at most −0.05, at most −0.1, at most −0.15, at most −0.2, at most −0.25, at most −0.3, at most −0.35, at most −0.4, at most −0.5, at most −0.6, at most −0.7, at most −0.8, at most −0.9, at most −1.0, at most −1.1, at most −1.2, at most −1.4, at most −1.5, at most −1.6, at most −1.8, or at most −2.0. In one or more embodiments, value “c” may be at least −5, such as at least −4, at least −3, or at least −2. In one or more preferred embodiments, value “c” may be from 0 to −1, such as −0.946.

In one or more embodiments, value “d” may be any value less than zero. In one or more of these embodiments, value “d” may be at most −0.01, at most −0.05, at most −0.1, at most −0.15, at most −0.2, at most −0.25, at most −0.3, at most −0.35, at most −0.4, at most −0.5, at most −0.6, at most −0.7, at most −0.8, at most −0.9, at most −1.0, at most −1.1, at most −1.2, at most −1.4, at most −1.5, at most −1.6, at most −1.8, or at most −2.0. In one or more embodiments, value “d” may be at least −5, such as at least −4, at least −3, or at least −2. In one or more preferred embodiments, value “d” may be from 0 to −1, such as −0.964.

In one or more embodiments, the value “X” may be any value of at least −10 to at most 10. In one or more of these embodiments, the value “X” may be from −9 to 9, from −8 to 8, from −7 to 7, from −6 to 6, from −5 to 5, from −4 to 4, from −3 to 3, from −2 to 2, or from −1 to 1. The value “X” may be from 0 to −5, from 0 to −4, from 0 to −3, or from 0 to −2. In one or more preferred embodiments, the value “X” may be from −4 to −3, such as −3.43.

While this risk score would typically equal the estimated probability of ovarian cancer, the intercept estimate is biased given the case-control study design and, thus, it is referred to herein more generally as a “risk score.”

In one or more embodiments, a risk score calculated using a method of the present disclosure represents a percent risk of a patient having ovarian cancer. For example, a risk score of 100% may indicate that a patient has ovarian cancer, while a risk score of 0% may indicate that a patient does not have ovarian cancer. In one or more other embodiments, a risk score calculated using a method of the present disclosure represents a general risk factor, but not a percentage chance of a patient having ovarian cancer. For example, a risk score of 0.9 may indicate that a patient likely has ovarian cancer, while a risk score of 0.3 may indicate that a patient likely does not have ovarian cancer. It should be noted that a risk score typically does not represent an absolute chance, but more often represents a likelihood of a patient having ovarian cancer. For example, while a patient may have a risk score of 1.0, it may still be possible that the patient does not have ovarian cancer.

The positive weights for CA125 and HE4 indicate higher predicted likelihood of cancer for those with higher expression, while the negative weights for ITGAV and SEZ6L indicate lower predicted likelihood of cancer for those with higher expression. Neither ITGAV nor SEZ6L have previously been identified as early-stage ovarian cancer biomarkers, and the levels of both proteins were significantly decreased in sera from women with early-stage ovarian cancer compared to healthy controls (Table 2, FIG. 3A, bottom panels). When the t-SNE plots clustered for all 68 proteins were examined (FIG. 7B) by expression of the four proteins included in our multi-protein classifier, the majority of the cancer samples expressed high levels of CA125 and HE4 (FIG. 7C, 7D) and low levels of ITGAV and SEZ6L (FIG. 7E, 7F).

The ROC curves for the multi-protein classifier and each of the four individual proteins included in the classifier are shown in FIG. 3B and summarized in Table 4. Compared to CA125 alone, the four-protein classifier improved the AUC from 0.958 (95% CI: 0.928-0.982) to 0.974 (95% CI: 0.949-0.989). The sensitivity at 98% specificity of the multi-protein classifier was also improved from 0.810 (95% CI: 0.707-0.897) for CA125 alone to 0.862 (95% CI: 0.776-0.933) for the multi-protein classifier. The improvement in AUC by the multi-protein classifier compared to using CA125 alone was statistically significant (p=0.02).

TABLE 4
Area under the curve (AUC) and sensitivities at 95% and 98% specificity
(with 95% confidence intervals [CIs]) for the multi-protein classifier and the
individual protein components for Cohorts #1 and #2.

| | | Sensitivity at 95% | Sensitivity at 98% | |
|---|---|---|---|---|
| | AUC | Specificity | Specificity | p-value¹ |
| Cohort #1 | | | | |
| Multi-protein | 0.974 (0.949, 0.989) | 0.914 (0.852, 0.964) | 0.862 (0.776, 0.933) | — |
| CA125 | 0.958 (0.928, 0.982) | 0.879 (0.802, 0.940) | 0.810 (0.707, 0.897) | 0.02 |
| HE4 | 0.857 (0.808, 0.901) | 0.612 (0.526, 0.716) | 0.578 (0.302, 0.672) | <0.001 |
| ITGAV | 0.832 (0.783, 0.878) | 0.440 (0.302, 0.621) | 0.276 (0.164, 0.440) | <0.001 |
| SEZ6L | 0.764 (0.709, 0.816) | 0.310 (0.207, 0.448) | 0.164 (0.078, 0.310) | <0.001 |
| Cohort #2 | | | | |
| Multi-protein | 0.933 (0.909, 0.955) | 0.792 (0.708, 0.844) | 0.661 (0.526, 0.771) | |
| CA125 | 0.916 (0.886, 0.942) | 0.745 (0.672, 0.807) | 0.635 (0.536, 0.750) | <0.001 |
| HE4 | 0.882 (0.850, 0.912) | 0.620 (0.536, 0.703) | 0.516 (0.359, 0.630) | <0.001 |
| ITGAV | 0.700 (0.653, 0.746) | 0.266 (0.172, 0.354) | 0.109 (0.036, 0.224) | <0.001 |
| SEZ6L | 0.603 (0.554, 0.651) | 0.130 (0.068, 0.182) | 0.057 (0.016, 0.115) | <0.001 |

¹Comparing the AUC of multi-protein classifier to the AUC of the listed classifier.

Typically, the present disclosure relates to a protein classifier that includes values from more than one protein. In one or more embodiments, a multi-protein classifier of the present disclosure may include expression values from at least two, at least three, at least four, at least five, at least six, at least seven, at least eight, at least nine, at least ten, at least 11, at least 12, at least 13, at least 14, at least 15, at least 16, at least 17, at least 18, at least 19, at least 20, at least 21, at least 22, at least 23, at least 24, at least 25 or at least 26 proteins.

### Validation of the Multi-Protein Classifier for Early Stage Ovarian Cancer Using a New Cohort of Early Stage Ovarian Cancer Samples.

To validate the multi-protein classifier on an unrelated set of serum samples, 192 early-stage ovarian cancer samples and 467 healthy control samples from four different institutions were assembled as Cohort #2 (Table 1). Similar to Cohort #1, the majority of the serum samples were from women with HGSOC (40%), followed by the endometrioid subtype (26%), clear cell carcinoma (19%), and mucinous ovarian cancer (8%).

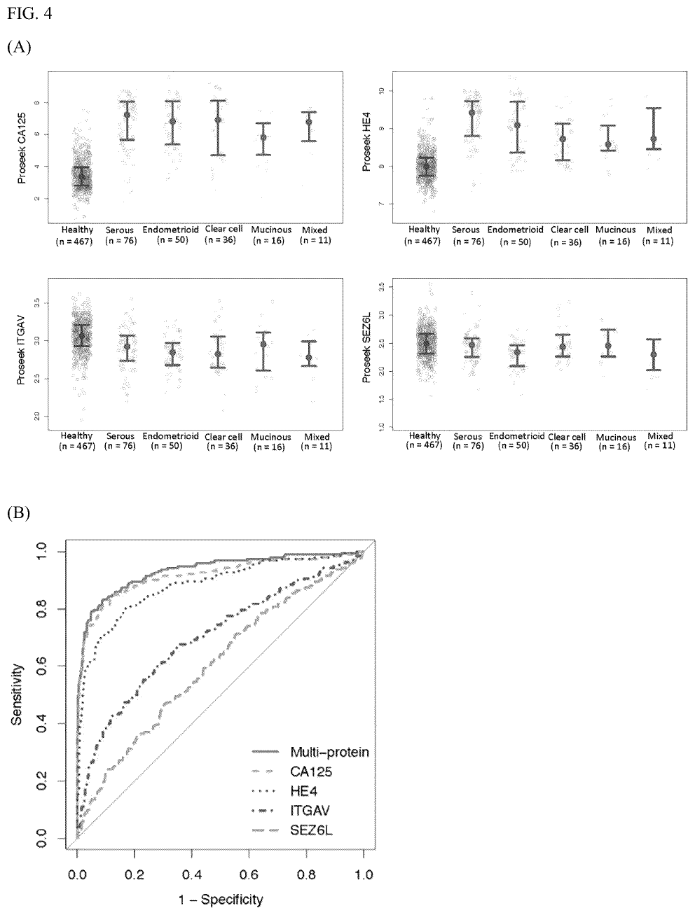

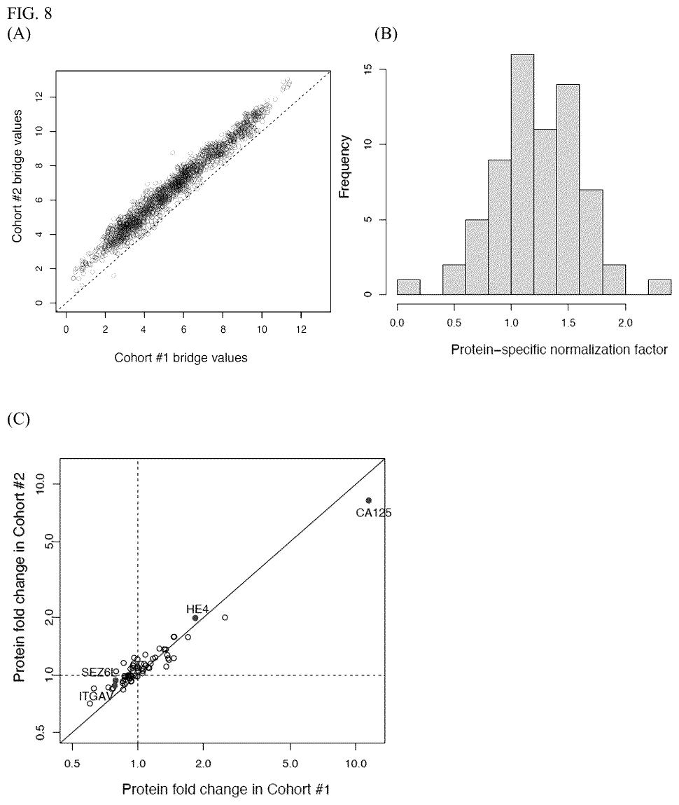

The NPX values between Cohort #1 and Cohort #2 were normalized using “bridge” samples from Cohort #1 (see Examples). The comparison of the NPX values prior to normalization for the bridge samples (across all proteins) is shown in FIG. 8A and a histogram of the protein-specific normalization factors is shown in FIG. 8B. The normalization factors for the four proteins of interest were 0.70 (CA125), 1.02 (HE4), 1.98 (ITGAV), and 1.95 (SEZ6L). When the NPX values for CA125 were compared to the clinical values for the ovarian cancer patients in Cohort #2, there was a correlation of 0.78, similar to what was observed in Cohort #1. The protein fold changes between ovarian cancer and healthy patients were similar for Cohort #1 and Cohort #2 (FIG. 8C). The NPX values in Cohort #2 for the four classifier proteins separated by ovarian cancer subtype are shown in FIG. 4A.

The early-stage multi-protein classifier was then applied to the Cohort #2 samples in order to validate its performance. The ROC curve for the early-stage multi-protein classifier applied to the Cohort #2 samples, along with the ROC curves for the four proteins individually, are shown in FIG. 4B and summarized in Table 4.

For the multi-protein classifier, the AUC was 0.933 (95% CI: 0.909-0.955). The sensitivity at 95% specificity was 0.792 (95% CI: 0.708-0.844) and the sensitivity at 98% specificity was 0.661 (95% CI: 0.526-0.771). For CA125 alone, the AUC was 0.916 (95% CI: 0.886-0.942). The modest improvement in AUC by the multi-protein classifier compared to using CA125 alone was statistically significant (p<0.001).

In one or more embodiments, a method of the present disclosure includes a classifier with an AUC of at least 0.80, at least 0.85, at least 0.90, at least 0.91, at least 0.92, at least 0.93, at least 0.94, at least 0.95, at least 0.96, at least 0.97, or at least 0.98, or at least 0.99. In one or more embodiments, a method of the present disclosure includes a multi-protein classifier with an AUC that is greater than a comparable single-protein classifier, such as a classifier using CA125 alone.

Validation of the Multi-Protein Classifier Using Serum Samples from Women with Benign Ovarian Conditions

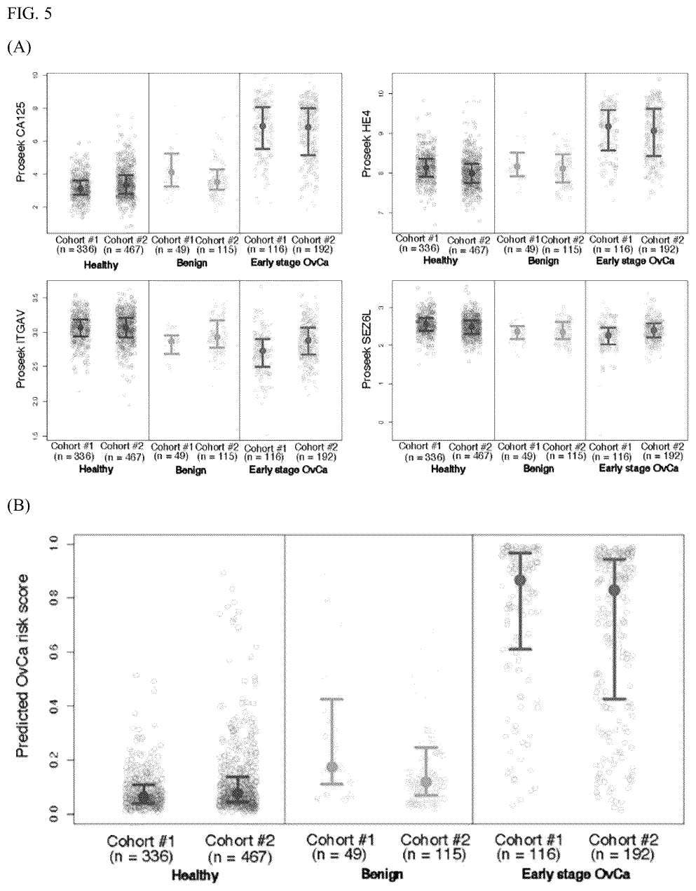

In order to examine the performance of our classifier in a broader sample set, the early-stage multi-protein classifier was applied to samples from women with benign ovarian conditions from the same institutions as Cohort #1 (n=49) and Cohort #2 (n=115). These serum samples were run on the PROSEEK Oncology II panel simultaneously with the ovarian cancer and healthy controls. The NPX expression levels for the four proteins in our multi-protein classifier are shown for ovarian cancer, benign, and healthy control samples from both cohorts in FIG. 5A. In general, the median NPX values for the benign samples were intermediate between those of the healthy controls and the ovarian cancer samples or similar to the NPX values from the healthy controls. The predicted cancer risk scores stratified by true cancer status are shown in FIG. 5B for both cohorts. Using the 98% specificity cutoff point, the multi-protein classifier correctly classified 80.5% of benign samples as “not cancer”.

Validation of the Multi-Protein Classifier for Early Stage Ovarian Cancer on Samples from Women with Late Stage Ovarian Cancer

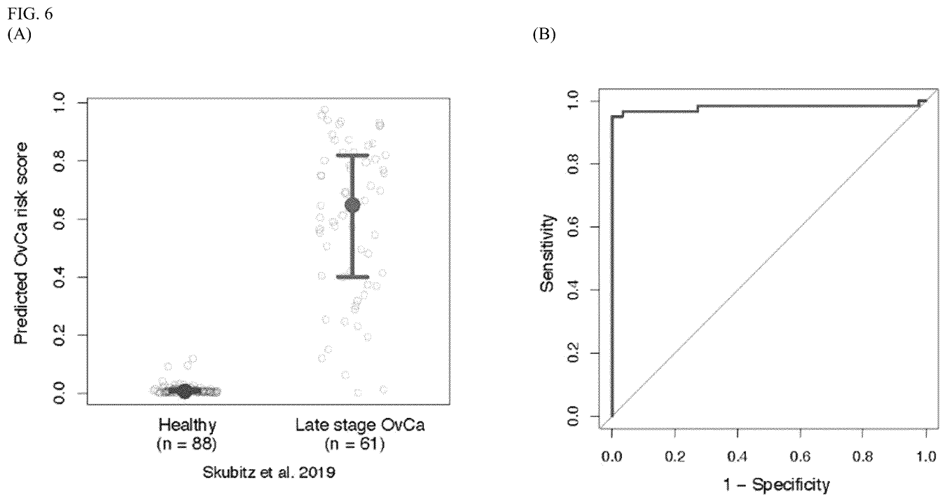

Protein changes in early-stage disease may not necessarily persist to later stage. To determine if the multi-protein classifier developed using the early-stage samples from Cohort #1 could retain its performance if presented with late-stage samples, the early-stage multi-protein classifier was applied to the NPX data from late-stage samples. Similar to what was observed in the early-stage samples, CA125 and HE4 levels were elevated in the late-stage ovarian cancer samples, while ITGAV and SEZ6L levels were higher in the healthy control samples. The predicted cancer risk scores, stratified by true cancer status, are shown in FIG. 6A for the late-stage samples. Given the lack of bridge samples between these two studies, one is unable to normalize between experiments and thus the risk scores are not directly comparable between studies. However, the multi-protein risk score discriminates the late-stage samples from the healthy controls. For the ROC curve for the late-stage samples using the early-stage multi-protein classifier (FIG. 6B), the AUC was 0.978 (95% CI: 0.941-1.00). The sensitivity at 95% specificity was 0.967 (95% CI: 0.902-1.00) and the sensitivity at 98% specificity was 0.951 (95% CI: 0.885-1.00). These data demonstrate that the multi-protein classifier developed using early-stage samples has strong discrimination performance between healthy and late-stage ovarian cancer samples.

Thus, in one aspect, this disclosure describes a multi-protein classifier that enables one to detect early and late stage ovarian. Specifically, the multi-protein classifier enables one to detect ovarian cancer before clear symptoms and/or clinical signs manifest in a patient, when ovarian cancer is more treatable. The multi-protein classifier uses protein levels in sera from early-stage ovarian cancer patients. By analyzing data from a cohort of 116 early-stage ovarian cancer and 336 healthy control patients from two institutions, the multi-protein classifier was developed to distinguish ovarian cancer cases from healthy controls. The classifier analyzes four proteins: CA125, HE4, ITGAV, and SEZ6L. When the four-protein classifier was tested with a validation cohort of 192 early-stage ovarian cancer and 467 healthy control patients from four different institutions, the four-protein classifier performed significantly better than CA125 alone. Of the 27 proteins that were significantly differentially expressed in both cohorts of serum, 11 proteins were found at decreased levels in ovarian cancer samples compared to the healthy controls, including the two proteins in the multi-protein classifier, ITGAV and SEZ6L.

ITGAV is a subunit of the alpha V family of integrins, that are involved in cell-cell and cell-matrix adhesions and signaling. High expression of ITGAV in ovarian cancer tumor tissue from late-stage tumors has been associated with poor prognosis. However, both tissue and serum levels of ITGAV have been shown to be present at reduced levels in ovarian cancer compared to benign tumors and borderline ovarian cancers. In addition, ITGAV expression has been correlated with increased expression of the matrix metalloprotease MMP9 in ovarian cancer effusions, which could affect ITGAV shedding into the serum in late-stage ovarian cancer.

The SEZ6L protein is a single pass transmembrane protein that may contribute to specialized endoplasmic reticulum functions. Genetic analyses have implicated the loss of SEZ6L gene function in the risk for development of lung cancer by deletion, and in colon cancer through promoter hypermethylation. Increased expression of SEZ6L in lung cancer cell lines and tumor tissues compared to normal lung cells suggest that SEZ6L is both a tumor biomarker and a genetic risk factor.

Including proteins expressed at lower levels in cancer compared to normal sera may seem somewhat counter-intuitive for a multi-protein classifier. Without wishing to be bound by any particular theory, there are multiple possible explanations for the ITGAV and SEZ6L being detected at lower levels in serum of ovarian cancer patients. One explanation could be that lower levels of proteins involved in immune response could result in reduced anti-tumor immunity in ovarian cancer patients. Indeed, 7 of the 11 proteins identified herein that were expressed at lower levels in sera from early-stage ovarian cancer patients than in the healthy controls play a role in immune response. Alternatively, antigen-autoantibody complex formation could mask the epitopes recognized by the protein quantification assay/platform, causing lower levels of protein to be detected in ovarian cancer patients. Another explanation could be that the proteins found at lower levels in ovarian cancer sera are more actively cleared and/or catabolized by the tumor-bearing host. However, for proteins present at very low levels in all samples, the difference may simply reflect the high degree of heterogeneity within the population, which can only be revealed by the inclusion of a large number of healthy control samples in the study. In the discovery cohort of the study described herein, a 3:1 ratio of control to ovarian cancer samples were included in the analysis in an attempt to address this issue. However, given the relatively low prevalence of ovarian cancer in the population, inclusion of even more control samples could improve classifier performance.

Although the multi-protein classifier described herein was developed using early-stage ovarian cancer samples compared to healthy controls, serum samples from women with benign ovarian conditions were run simultaneously. When the multi-protein classifier was applied to the 164 benign samples with a threshold of 98% specificity, 80.5% of the benign samples were classified correctly, with only ˜20% of the benign cases being classified as “cancer.” The multi-protein classifier could be incorporated into a two-step screening strategy whereby those women whose serum tests indicate “cancer” would then be screened by imaging to rule out the false-positive benign lesions and exclude them from surgery.

While multi-protein classifiers using CA125 in addition to one, two, or three additional proteins, such as SEZ6L, HE4, and ITGAV are shown herein to be efficacious, inclusion of additional proteins may improve sensitivity.

Some proteins are identified herein to be expressed at elevated levels in patients with ovarian cancer. Proteins expressed at elevated levels include MK (UniProt ID No.: P21741), IL6 (UniProt ID No.: P05231), ESM-1 (UniProt ID No.: Q9NQ30), hK11 (UniProt ID No.: Q9UBX7), ADAM-TS 15 (UniProt ID No.: Q8TE58), SYND1 (UniProt ID No.: P18827), CXCL13 (UniProt ID No.: O43927), TFPI-2 (UniProt ID No.: P48307), FR-α (UniProt ID No.: P15328), KLK13 (UniProt ID No.: Q9UKR3), MSLN (UniProt ID No.: Q13421), NECT4 (UniProt ID No.: Q96NY8), TNFRSF6B (UniProt ID No.: O95407), FCRLB (UniProt ID No.: Q6BAA4), and AREG (UniProt ID No.: P15514). In one or more embodiments, a method may include providing a protein level of one or more proteins of these proteins and identifying a patient with above normal levels of the one or more proteins.

Some proteins are identified herein to be expressed at decreased levels in patients with ovarian cancer. Proteins expressed at decreased levels include SCF (UniProt ID No.: P21583), FASLG (UniProt ID No.: P48023), XPNPEP2 (UniProt ID No.: O43895), TCL1A (UniProt ID No.: P56279), VEGFR-2 (UniProt ID No.: P35968), CEACAM1 (UniProt ID No.: P13688), TLR3 (UniProt ID No.: O15455), CYR61 (UniProt ID No.: O00622), GPNMB (UniProt ID No.: Q14956), CPE (UniProt ID No.: P16870), LY9 (UniProt ID No.: Q9HBG7), ERBB2 (UniProt ID No.: P04626), GPC1 (UniProt ID No.: P35052), IFN-γ-R1 (UniProt ID No.: P15260), CD48 (UniProt ID No.: P09326), RET (UniProt ID No.: P07949), ICOSLG (UniProt ID No.: O75144), CTSV (UniProt ID No.: O60911), and MIA (UniProt ID No.: Q16674). In one or more embodiments, a method may include providing a protein level of one or more proteins of these proteins and identifying a patient with below normal levels of the one or more proteins. As it is used herein, “providing” a level of a protein includes utilizing known information about the level of that protein or measuring a level of the protein in a sample.

In one or more embodiments, measuring a level of a protein in a sample includes detecting the protein using an antibody. Methods of detecting proteins using antibodies are often referred to as immunoassays. Suitable immunoassays include enzyme-linked immunosorbent assays, enzyme multiplied immunoassay techniques, DNA-based methods, such as immunoquantitative PCR (immunoPCR), electrochemiluminescent (ECL) assays, and radioactive reporter assays. One preferred method of measuring protein levels is the Olink proximity extension assay (PEA), which utilizes an oligonucleotide-antibody pair to detect and amplify proteins. It should be recognized that some proteins may be present in a sample at very low levels, and thus, a sensitive detection method may be appropriate.

The multi-protein classifier described herein uses ovarian cancer biomarkers that are robust and not sensitive to preanalytical variation. The multi-protein classifier includes protein biomarkers that are present at reduced levels in early-stage ovarian cancer cases compared to healthy controls. The data presented herein show that including biomarkers with reduced levels in early-stage ovarian cancer cases can increase the predictive value of a multi-protein classifier, suggesting that the lower levels of some proteins may contribute to tumor development.

The multi-protein classifier described herein may be supplemented with other biomarkers indicative of early-stage ovarian cancer including, but not limited to, autoantibodies, circulating tumor DNA, miRNA, cell-free DNA, cancer-associated metabolites, circulating tumor cells, immune factors, microbial proteins, or other molecules. The methods described herein may be supplemented with other factors indicative of early-stage ovarian cancer, such as age, menopausal status, weight, body mass index, familial history of cancer, smoker status, stress level, and activity level.

In one or more embodiments, a method includes treating a patient for ovarian cancer. A patient may be treated if a calculated risk score exceeds a certain threshold. A method may include supplementing a multi-protein classifier described herein with additional information and determining whether to treat the patient using the information provided by the multi-protein classifier and additional information. For example, a multi-protein classifier described herein may be used to identify a patient having abnormal serum protein levels of more than one protein associated with ovarian cancer. The patient may further be subjected to an ultrasound to detect solid masses within or proximal to the ovaries. The patient may be diagnosed with and treated for ovarian cancer. Treatments for ovarian cancer are known in the art and include surgery, immunotherapy, radiation therapy, and chemotherapy.

In another aspect, the present disclosure relates to a kit. A kit may include reagents and instructions to analyze a sample from a patient for levels of one or more proteins and calculate a risk factor for the patient, wherein the risk factor indicates whether the patient has ovarian cancer.

In one or more embodiments, a kit includes a first reagent to measure a level of CA125 protein, a second reagent to measure the level of HE4 protein, a third reagent to measure a level of ITGAV, and a fourth reagent to measure a level of SEZ6L in a biological sample. Each of the first, second, third, and fourth reagents may be the same class of biomolecule (e.g., protein, oligonucleotide, lipid). In one or more preferred embodiments, each of the first, second, third, and fourth reagents includes an antibody and an oligonucleotide. Each reagent may include more than one antibody, such as two antibodies.

In one or more embodiments, the kit includes a plate including wells. In one or more particular embodiments, a first well includes the first reagent, a second well includes the second reagent, a third well includes the third reagent, and a fourth well includes the fourth reagent. In one or more alternative embodiments, each of the first, second, third, and fourth reagents may be present in the same well. The plate may have any suitable number of wells, such as one, four, six, 12, 24, 48, or 96 wells.

Optionally, other reagents such as buffers and solutions needed to use the cytidine deaminase and nucleotide solution are also included. Instructions for use of the kit components are also typically included.

In another aspect, the present disclosure relates to a tangible and/or non-transitory computer readable media or computer program products that include instructions and/or data (including data structures) for performing various computer-implemented operations. One or more of the steps of a method set forth herein can be carried out by a computer program that is present in tangible and/or non-transitory computer readable media, or carried out using computer hardware.

For example, a computer program product is provided and it comprises a non-transitory computer readable medium on which is provided program instructions for providing levels of CA125 protein, HE4 protein, ITGAV protein, and SEZ6L protein from at least one reference sample; determining, for each protein, a difference in the level of protein in the biological sample and the reference sample, thereby providing a normalized level of each protein; weighting the normalized level of CA125 using a first coefficient “a”, wherein “a” is a positive value; weighting the normalized level of HE4 using a second coefficient “b”, wherein “b” is a positive value; weighting the normalized level of ITGAV using a third coefficient “c”, wherein “c” is a negative value; weighting the normalized level of SEZ6L using a fourth coefficient “d”, wherein “d” is a negative value; determining an intercept value X; and determining an ovarian cancer risk score for the patient, wherein the ovarian cancer risk score is calculated using Formula II:

$$Risk score \% = 100 * ( \frac{{e}^{X + a ( CA 125 ) + b ( HE 4 ) + c ( ITGAV ) + d ( SEZ 6 L )}}{1 + {e}^{X + a ( CA 125 ) + b ( HE 4 ) + c ( ITGAV ) + d ( SEZ 6 L )}} ) \qquad Formula II$$

wherein the ovarian cancer risk score indicates a percent chance the patient has ovarian cancer.

In one or more embodiments, a coefficient, such as coefficient a, b, c, or d, is determined using the level of a protein in a biological sample and a level of the same protein in a reference sample.

In one example, a user provides a sample analysis device, such as a real-time PCR system. Data is collected and/or analyzed by the device which is connected to a computer. Software on the computer allows for data collection and/or analysis. Data can be stored, displayed (e.g. via a monitor or other similar device), and/or sent to another location. The computer may be connected to the internet which is used to transmit data to a handheld device and/or cloud environment utilized by a remote user (e.g., a physician, scientist, or analyst). It is understood that the data can be stored and/or analyzed prior to transmittal. In one or more embodiments, raw data is collected and sent to a remote user or apparatus that will analyze and/or store the data. Transmittal can occur via the internet, but can also occur via satellite or other connection. Alternatively, data can be stored on a computer-readable medium and the medium can be shipped to an end user (e.g., via mail). The remote user can be in the same or a different geographical location including, but not limited to, a building, city, state, country, or continent.

In one or more embodiments, the methods also include collecting data regarding levels of a plurality of proteins and sending the data to a computer or other computational system. For example, the computer can be connected to laboratory equipment, e.g., a sample collection apparatus or a plate reader. The computer can then collect applicable data gathered by the laboratory device. The data can be stored on a computer at any step, e.g., while collected in real time, prior to the sending, during or in conjunction with the sending, or following the sending. The data can be stored on a computer-readable medium that can be extracted from the computer. The data that has been collected or stored can be transmitted from the computer to a remote location, e.g., via a local network or a wide area network such as the internet. At the remote location various operations can be performed on the transmitted data as described below.

Data regarding protein levels in a biological sample may be obtained, stored, transmitted, analyzed, and/or manipulated at one or more locations using distinct apparatus. The processing options span a wide spectrum. Toward one end of the spectrum, all or much of this information is stored and used at the location where the test sample is processed, e.g., a doctor's office or other clinical setting. Toward another extreme, the sample is obtained at one location, it is processed (e.g. prepared and/or detected) at a second location, data is analyzed (e.g. protein levels are run through an algorithm), and diagnoses, recommendations, and/or plans are prepared at a fourth location (or the location where the sample was obtained). The methods described herein may be carried out regardless of where a sample is obtained, where it is processed, where the data is analyzed, and where diagnoses are set forth. As the methods described herein relate to analysis of a provided sample, use of an algorithm as described herein independently, i.e., separately from sample collection and diagnosis, is encompassed by the present disclosure.

In one or more embodiments, the present disclosure relates to a system to perform one or more of the methods described herein. A system may include, for example, a kit including reagents, a device, and software programming to analyze results.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

The present invention is illustrated by the following examples. It is to be understood that the particular examples, materials, amounts, and procedures are to be interpreted broadly in accordance with the scope and spirit of the invention as set forth herein.

### EXEMPLARY EMBODIMENTS

Embodiment 1 is a method for determining risk of ovarian cancer in a patient, the method comprising:
providing a biological sample from the patient;
measuring a level of mucin 16 (CA125) in the biological sample;
measuring a level of seizure 6-like protein (SEZ6L) in the biological sample; and
identifying that the patient is at risk of ovarian cancer based on the level of CA125 and the level of SEZ6L.

Embodiment 2 is the method of Embodiment 1, wherein identifying comprises:
identifying a normal level of CA125;
comparing the level of CA125 measured in the biological sample to the normal level of CA125;
identifying that the level of CA125 measured in the biological sample is greater than the normal level of CA125;
identifying a normal level of SEZ6L;
comparing the level of SEZ6L measured in the biological sample to the normal level of SEZ6L; and
identifying that the level of SEZ6L measured in the biological sample is less than the normal level of SEZ6L.

Embodiment 3 is the method of Embodiment 1, further comprising:
measuring a level of human epididymis protein 4 (HE4) in the biological sample;
measuring a level of integrin alpha-V (ITGAV) in the biological sample; and
identifying that the patient is at risk of ovarian cancer based on the levels of CA125, SEZ6L, HE4, and ITGAV.

Embodiment 4 is the method of Embodiment 3, wherein identifying comprises:
identifying a normal level of HE4;
comparing the level of HE4 measured in the biological sample to the normal level of HE4;
identifying that the level of HE4 measured in the biological sample is greater than the normal level of HE4;
identifying a normal level of ITGAV;
comparing the level of ITGAV measured in the biological sample to the normal level of ITGAV; and
identifying that the level of ITGAV measured in the biological sample is less than the normal level of ITGAV.

Embodiment 5 is the method of any preceding Embodiment, wherein the biological sample comprises serum, whole blood, plasma, saliva, urine, mucus, ascites fluid, cervical swabs, vaginal swabs, fine needle aspirates, and/or biopsied cells.

Embodiment 6 is a method for determining an ovarian cancer risk score in a patient comprising:
providing a biological sample;
measuring a level of CA125 protein, a level of protein, a level of ITGAV protein, and a level of SEZ6L protein in the biological sample;
providing a level of CA125 protein, a level of HE4 protein, a level of ITGAV protein, and a level of SEZ6L protein from at least one reference sample;
determining, for each protein, a difference in the level of protein in the biological sample and the reference sample, thereby providing a normalized level of each protein;
weighting the normalized level of CA125 using a first coefficient a, wherein a is a positive value;
weighting the normalized level of HE4 using a second coefficient b, wherein b is a positive value;
weighting the normalized level of using a third coefficient c, wherein c is a negative value;
weighting the normalized level of SEZ6L using a fourth coefficient d, wherein d is a negative value;
determining an intercept value X; and
determining an ovarian cancer risk score for the patient, wherein the ovarian cancer risk score is calculated using Formula II:

$$Risk score \% = 100 * ( \frac{{e}^{X + a ( CA 125 ) + b ( HE 4 ) + c ( ITGAV ) + d ( SEZ 6 L )}}{1 + {e}^{X + a ( CA 125 ) + b ( HE 4 ) + c ( ITGAV ) + d ( SEZ 6 L )}} ) \qquad Formula II$$

wherein the ovarian cancer risk score indicates a percent chance the patient has ovarian cancer.

Embodiment 7 is the method of any preceding Embodiment, further comprising treating the patient for ovarian cancer.

Embodiment 8 is the method of Embodiment 6, wherein a is a value from 0 to 2.

Embodiment 9 is the method of Embodiment 6, wherein b is a value from 0 to 2.

Embodiment 10 is the method of Embodiment 6, wherein c is a value from −2 to 0.

Embodiment 11 is the method of Embodiment 6, wherein d is a value from −2 to 0.

Embodiment 12 is the method of Embodiment 6, wherein X is a value from −5 to 5.

Embodiment 13 is the method of Embodiment 6, wherein the ovarian cancer risk score is calculated using Formula I:

$$Risk score \% = 100 * ( \frac{{e}^{- 3.43 + 0.959 ( CA 125 ) + 0.38 ( HE 4 ) + ( - 0.946 ) ( ITGAV ) + ( - 0.964 ) ( SEZ 6 L )}}{1 + {e}^{- 3.43 + 0.959 ( CA 125 ) + 0.38 ( HE 4 ) + ( - 0.946 ) ( ITGAV ) + ( - 0.964 ) ( SEZ 6 L )}} ) . \qquad Formula I$$

Embodiment 14 is the method of Embodiment 6, wherein the biological sample comprises serum, whole blood, plasma, saliva, urine, mucus, ascites fluid, cervical swabs, vaginal swabs, fine needle aspirates, and/or biopsied cells.

Embodiment 15 is the method of any preceding Embodiment, wherein measuring protein levels in the biological sample comprises an immunoassay.

Embodiment 16 is the method of any preceding Embodiment, wherein measuring protein levels in the biological sample comprises polymerase chain reaction (PCR).

Embodiment 17 is the method of any preceding Embodiment, wherein measuring protein levels in the biological sample comprises immuno-PCR, such as proximity extension assay qPCR.

Embodiment 18 is the method of any preceding Embodiment, further comprising analyzing the biological sample for one or more additional indicators of ovarian cancer, the indicator of ovarian cancer comprising an autoantibody, a metabolite, a cell-free DNA, a circulating tumor cell, a molecule of circulating tumor DNA, an immune factor, an miRNA, microbial protein or DNA, or another indicator of ovarian cancer.

Embodiment 19 is the method of any preceding Embodiment, further comprising analyzing the age, menopausal status, weight, body mass index, familial history of cancer, smoker status, stress level, and/or activity level.

Embodiment 20 is the method of any preceding Embodiment, wherein the ovarian cancer is early-stage ovarian cancer.

Embodiment 21 is the method of Embodiment 20, wherein the early-stage ovarian cancer is American Joint Committee on Cancer Stage I.

Embodiment 22 is the method of Embodiment 20, wherein the ovarian cancer comprises clear cell ovarian cancer, mucinous ovarian cancer, or endometroid ovarian cancer.

Embodiment 23 is the method of any preceding Embodiment, wherein the ovarian cancer comprises high grade serous ovarian cancer (HGSOC),

Embodiment 24 is the method of any preceding Embodiment, wherein the method demonstrates higher sensitivity at a set specificity as compared to a method wherein only CA125 is analyzed.

Embodiment 25 is the method of any preceding Embodiment, further comprising:
providing a protein level of one or more of SCF (UniProt ID No.: P21583), FASLG (UniProt ID No.: P48023), XPNPEP2 (UniProt ID No.: O43895), TCL1A (UniProt ID No.: P56279), VEGFR-2 (UniProt ID No.: P35968), CEACAM1 (UniProt ID No.: P13688), TLR3 (UniProt ID No.: O15455), CYR61 (UniProt ID No.: O00622), GPNMB (UniProt ID No.: Q14956), CPE (UniProt ID No.: P16870), LY9 (UniProt ID No.: Q9HBG7), ERBB2 (UniProt ID No.: P04626), GPC1 (UniProt ID No.: P35052), IFN-γ-R1 (UniProt ID No.: P15260), CD48 (UniProt ID No.: P09326), RET (UniProt ID No.: P07949), ICOSLG (UniProt ID No.: O75144), CTSV (UniProt ID No.: O60911), and MIA (UniProt ID No.: Q16674); and
identifying a patient with below normal levels of the one or more proteins as at risk for ovarian cancer.

Embodiment 26 is the method of Embodiment 24, wherein providing comprises measuring.

Embodiment 27 is the method of any preceding Embodiment, further comprising:
providing a protein level of one or more of MK (UniProt ID No.: P21741), IL6 (UniProt ID No.: P05231), ESM-1 (UniProt ID No.: Q9NQ30), hK11 (UniProt ID No.: Q9UBX7), ADAM-TS 15 (UniProt ID No.: Q8TE58), SYND1 (UniProt ID No.: P18827), CXCL13 (UniProt ID No.: O43927), TFPI-2 (UniProt ID No.: P48307), FR-α (UniProt ID No.: P15328), KLK13 (UniProt ID No.: Q9UKR3), MSLN (UniProt ID No.: Q13421), NECT4 (UniProt ID No.: Q96NY8), TNFRSF6B (UniProt ID No.: O95407), FCRLB (UniProt ID No.: Q6BAA4), and AREG (UniProt ID No.: P15514); and
identifying a patient with above normal levels of the one or more proteins as at risk for ovarian cancer.

Embodiment 28 is the method of any preceding Embodiment, wherein providing comprises measuring.

Embodiment 29 is the method of any preceding Embodiment, wherein the levels of at most 25 proteins are measured.

Embodiment 30 is a kit comprising:
a first reagent to measure the level of CA125 protein in a biological sample, the first reagent comprising an antibody and an oligonucleotide;
a second reagent to measure the level of HE4 protein in a biological sample, the second reagent comprising an antibody and an oligonucleotide;
a third reagent to measure the level of ITGAV protein in a biological sample, the third reagent comprising an antibody and an oligonucleotide; and
a fourth reagent to measure the level of SEZ6L protein in a biological sample, the fourth reagent comprising an antibody and an oligonucleotide.

Embodiment 31 is the kit of Embodiment 30, wherein each of the first, second, third, and fourth reagents comprises an antibody and an oligonucleotide.

Embodiment 32 is the kit of Embodiment 30, further comprising a reference sample.

Embodiment 33 is the kit of Embodiment 30, further comprising a multi-well plate.

Embodiment 34 is a system for performing the method of Embodiment 1.

Embodiment 35 is a system for performing the method of Embodiment 6.

Embodiment 36 is a computer program comprising a non-transitory computer readable medium on which is provided program instructions for steps of
providing a level of CA125 protein, a level of HE4 protein, a level of ITGAV protein, and a level of SEZ6L protein from a biological sample from a patient;
providing a level of CA125 protein, a level of ELEA protein, a level of ITGAV protein, and a level of SEZ6L protein from at least one reference sample;
determining, for each protein, a difference in the level of protein in the biological sample and the reference sample, thereby providing a normalized level of each protein;
weighting the normalized level of CA125 using a first coefficient a, wherein a is a positive value;
weighting the normalized level of HE4 using a second coefficient b, wherein is a positive value;
weighting the normalized level of ITGAV using a third coefficient c, wherein c is a negative value;
weighting the normalized level of SEZ6L using a fourth coefficient d, wherein d is a negative value;
determining an intercept value X; and
determining an ovarian cancer risk score for the patient, wherein the ovarian cancer risk score is calculated using Formula II:

$$Risk score \% = 100 * ( \frac{{e}^{X + a ( CA 125 ) + b ( HE 4 ) + c ( ITGAV ) + d ( SEZ 6 L )}}{1 + {e}^{X + a ( CA 125 ) + b ( HE 4 ) + c ( ITGAV ) + d ( SEZ 6 L )}} ) \qquad Formula II$$

wherein the ovarian cancer risk score indicates a percent chance the patient has ovarian cancer.

### EXAMPLES

### Serum Samples

Blood samples were collected prior to treatment (surgery or chemotherapy) from women diagnosed with stage I-II epithelial ovarian cancer of all subtypes, benign ovarian conditions, or age-matched healthy controls under IRB approved protocols. Cohort #1 samples were collected at the University of Minnesota (Minneapolis, MN) and M.D. Anderson Cancer Center (Houston, TX). These samples were used in the discovery phase of experiments to develop a multi-protein classifier. Cohort #2 samples were collected at the Brigham Women's Hospital, Harvard Medical School (Boston, MA), Fox Chase Cancer Center (Philadelphia, PA), European Institute of Oncology (Milan, Italy), and Oregon Health & Science University (Portland, OR). These samples were used for the validation phase of experiments. All samples were collected after obtaining consent using IRB approved protocols.

### OLINK PROSEEK Oncology II Assay

The levels of 92 oncology related proteins were quantified in 1 μl of serum using the PROSEEK Oncology II proximity extension immunoassay panel (Olink Proteomics, Uppsala, Sweden) as previously described (Skubitz et al. Cancer Prev Res 12(3):171-184, 2019). Samples were randomly assigned to 96-well plates using stratified randomization based on institution of origin, diagnosis (healthy vs. cancer), ovarian cancer subtype, age, and race (when available). Samples were run on the PROSEEK Oncology II panel to quantify the level of protein expression. Each sample was mixed with the PROSEEK Oncology II reagents according to the manufacturer's instructions and quantified by qPCR using a high-throughput PCR instrument (BIOMARK HD, Standard BioTools, Inc., South San Francisco, CA) at the UNIVERSITY OF MINNESOTA Genomics Center. The PROSEEK platform includes three “interplate controls” for data normalization between plates and three “negative controls” to establish background levels. Internal controls for incubation and extension are included by Olink in each assay for quality control. The PROSEEK assay reports relative quantification on a log 2 scale, as Normalized Protein eXpression (NPX) values, which was normalized according to the manufacturer's protocols. Samples that did not pass Olink quality control were not included in the analysis.

### Identification of Unstable Proteins

In Cohort #1, case-control differences between institutions were explored by fitting a linear regression model with an interaction term of institution (MN vs. TX) and disease status (ovarian cancer vs. healthy), along with the corresponding main effects, for each protein with a Holm's adjustment to account for multiple testing. Proteins whose case-control differences varied significantly between institutions were excluded due to concern that these proteins' levels were unstable, meaning too sensitive to preanalytical conditions (e.g., environmental factors such as pre-processing storage time or the pre- or post-centrifugation temperatures). Findings from Cohort #1 were compared to previous work (Shen et al., Clin Chem Lab Med. 56(4):582-594, 2018) that investigated the impact of environmental factors on quantified protein levels for Olink panels for cardiovascular disease (Olink CVD I) and inflammation (Olink Inflammation I).

### Data Normalization for Cohort #2

Twenty-two “bridge” samples (12 ovarian cancer and 10 healthy controls; 2-3 samples per 96-well plate) were used to normalize data between Cohort #1 and Cohort #2 using Olink's recommended approach (https://www.olink.com/question/how-can-i-compare-results-from-two-different-studies/). Specifically, differences in NPX values were calculated between the “bridge” samples from Cohort #1 and Cohort #2, and then the median of these pairwise differences were calculated for each protein, referred to herein as the “normalization factor.” The NPX values for each of the proteins for samples in Cohort #2 were normalized by subtracting the protein-specific normalization factor. This data normalization is necessary since the PROSEEK assay reports relative (vs. absolute) quantification.

### Statistical Analysis

The data were normalized by the UNIVERSITY OF MINNESOTA Genomics Center, per the manufacturer's protocol. Differences in mean expression between cancer and healthy samples were determined using two-sample t-tests assuming unequal variances with p-values adjusted to control the false discovery rate at 5%. Single-protein classification accuracy was evaluated using the empirical receiver operating characteristic (ROC) curve and was summarized by the area under the ROC curve (AUC) and the sensitivities corresponding to specificities of 0.95 and 0.98 (i.e., ROC (0.05) and ROC (0.02), respectively). To summarize the value added beyond the contribution of CA125, the same summaries (AUC, ROC (0.05), and ROC (0.02)) were calculated for two-protein classifiers that included CA125 and one other protein. These two-protein classifiers were fit on Cohort #2 using a previously described method (Meisner et al., Biom J. 63(6):1223-1240, 2021) to maximize the sensitivity for a fixed specificity of 0.95 and assessed on Cohort #1. Confidence intervals (CIs) for AUC, ROC (0.05), and ROC (0.02) were calculated using a non-parametric bootstrap approach.

A multi-protein classifier was developed to differentiate healthy controls from early-stage ovarian cancer cases using least absolute shrinkage and selection operator (LASSO) logistic regression with the tuning parameter chosen using 10-fold cross-validation to be that with cross-validation error within 1 standard error of the minimum cross-validation error (“lambda.1se”, Tibshirani R., Journal of the Royal Statistical Society Series B 58(1):267-88, 1996). Summaries of the classification accuracy were estimated using the predicted probabilities from the held-out cross-validation folds. To obtain CIs for AUC, ROC (0.05), and ROC (0.02) for the multi-protein classifier, the bias-corrected bootstrap case cross-validation method of Jiang et al. (Stat Appl Genet Mol Biol. 7(1):Article8, 2008) was used. The difference in AUCs between different classifiers was tested using a bootstrap method for correlated ROC curves. All analyses were performed in R version 4.0.2 (R Foundation for Statistical Computing, Vienna, Austria) using the R packages glmnet (Friedman et al., J Stat Softw. 33(1):1-22, 2010), maxTPR (Meisner A., Maximizing the TPR for a specified FPR. R package version 0.1.0.2017, https://CRAN.R-project.org/package=maxTPR), and pROC (Robin et al., BMC Bioinformatics. 12:77, 2011).

### Unsupervised Hierarchical Clustering Analysis

Unsupervised clustering methods were applied to the data to identify clusters of proteins and visually evaluate their association with disease status. Unsupervised hierarchical clustering (uncentered correlation using centroid linkage) was completed using Cluster 3.0 (de Hoon et al., Bioinformatics. 20(9):1453-1454, 2004) and visualized using Treeview (v1.1.6r4). Principal component analysis (PCA) was performed using the prcomp function in R and t-distributed Stochastic Neighbor Embedding (t-SNE) was done using the Rtsne package in R.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method for determining risk of ovarian cancer in a patient, the method comprising:
providing a biological sample from the patient;
measuring a level of mucin 16 (CA125) in the biological sample;
measuring a level of seizure 6-like protein (SEZ6L) in the biological sample; and
identifying that the patient is at risk of ovarian cancer based on the level of CA125 and the level of SEZ6L.

2. The method of claim 1, wherein identifying comprises:
identifying a normal level of CA125;
comparing the level of CA125 measured in the biological sample to the normal level of CA125;
identifying that the level of CA125 measured in the biological sample is greater than the normal level of CA125;
identifying a normal level of SEZ6L;
comparing the level of SEZ6L measured in the biological sample to the normal level of SEZ6L; and
identifying that the level of SEZ6L measured in the biological sample is less than the normal level of SEZ6L.

3. The method of claim 1, further comprising:
measuring a level of human epididymis protein 4 (HE4) in the biological sample;
measuring a level of integrin alpha-V (ITGAV) in the biological sample; and
identifying that the patient is at risk of ovarian cancer based on the levels of CA125, SEZ6L, HE4, and ITGAV.

4. The method of claim 3, wherein identifying comprises:
identifying a normal level of HE4;
comparing the level of HE4 measured in the biological sample to the normal level of HE4;
identifying that the level of HE4 measured in the biological sample is greater than the normal level of HE4;
identifying a normal level of ITGAV;
comparing the level of ITGAV measured in the biological sample to the normal level of ITGAV; and
identifying that the level of ITGAV measured in the biological sample is less than the normal level of ITGAV.

5. The method of claim 1, wherein the biological sample comprises serum, whole blood, plasma, saliva, urine, mucus, ascites fluid, a cervical swab, a vaginal swab, fine needle aspirate, and/or biopsied cells.

6. A method for determining an ovarian cancer risk score in a patient comprising:
providing a biological sample;
measuring a level of CA125 protein, a level of HE4 protein, a level of ITGAV protein, and a level of SEZ6L protein in the biological sample;
providing levels of CA125 protein, HE4 protein, ITGAV protein, and SEZ6L protein from at least one reference sample;
determining, for each protein, a difference in the level of protein in the biological sample and the reference sample, thereby providing a normalized level of each protein;
weighting the normalized level of CA125 using a first coefficient a, wherein a is a positive value;
weighting the normalized level of H1 using a second coefficient b, wherein b is a positive value;
weighting the normalized level of ITGAV using a third coefficient c, wherein c is a negative value;
weighting the normalized level of SEZ6L using a fourth coefficient d, wherein d is a negative value;
determining an intercept value X; and
determining an ovarian cancer risk score for the patient, wherein the ovarian cancer risk score is calculated using Formula II:
$$Risk score \% = 100 * ( \frac{{e}^{X + a ( CA 125 ) + b ( HE 4 ) + c ( ITGAV ) + d ( SEZ 6 L )}}{1 + {e}^{X + a ( CA 125 ) + b ( HE 4 ) + c ( ITGAV ) + d ( SEZ 6 L )}} ) \qquad Formula II$$
wherein the ovarian cancer risk score indicates a percent chance the patient has ovarian cancer.

7. The method of claim 6, further comprising treating the patient for ovarian cancer.

8. The method of claim 6, wherein a is a value from 0 to 2, b is a value from 0 to 2, c is a value from −2 to 0, d is a value from −2 to 0, and X is a value from −5 to 5.

9. The method of claim 6, wherein the ovarian cancer risk score is calculated using Formula I:
$$Risk score \% = 100 * ( \frac{{e}^{- 3.43 + 0.959 ( CA 125 ) + 0.38 ( HE 4 ) + ( - 0.946 ) ( ITGAV ) + ( - 0.964 ) ( SEZ 6 L )}}{1 + {e}^{- 3.43 + 0.959 ( CA 125 ) + 0.38 ( HE 4 ) + ( - 0.946 ) ( ITGAV ) + ( - 0.964 ) ( SEZ 6 L )}} ) . \qquad Formula I$$

10. The method of claim 6, wherein the biological sample comprises serum, whole blood, plasma, saliva, urine, mucus, and/or biopsied cells.

11. The method of claim 1, wherein the ovarian cancer is early-stage ovarian cancer.

12. The method of claim 11, wherein the ovarian cancer comprises clear cell ovarian cancer, mucinous ovarian cancer, or endometroid ovarian cancer.

13. The method of claim 1, wherein the method demonstrates higher sensitivity at a set specificity as compared to a method wherein only CA125 is analyzed.

14. The method of claim 1, further comprising:
providing a protein level of one or more of SCF (UniProt ID No.: P21583), FASLG (UniProt ID No.: P48023), XPNPEP2 (UniProt ID No.: 043895), TCL1A (UniProt ID No.: P56279), VEGFR-2 (UniProt ID No.: P35968), CEACAM1 (UniProt ID No.: P13688), TLR3 (UniProt ID No.: O15455), CYR61 (UniProt ID No.: O00622), GPNMB (UniProt ID No.: Q14956), CPE (UniProt ID No.: P16870), LY9 (UniProt ID No.: Q9HBG7), ERBB2 (UniProt ID No.: P04626), GPC1 (UniProt ID No.: P35052), IFN-γ-R1 (UniProt ID No.: P15260), CD48 (UniProt ID No.: P09326), RET (UniProt ID No.: P07949), ICOSLG (UniProt ID No.: O75144), CTSV (UniProt ID No.: O60911), and MIA (UniProt ID No.: Q16674); and
identifying a patient with below normal levels of the one or more proteins as at risk for ovarian cancer.

15. The method of claim 1, further comprising:
providing a protein level of one or more of MK (UniProt ID No.: P21741), IL6 (UniProt ID No.: P05231), ESM-1 (UniProt ID No.: Q9NQ30), hK11 (UniProt ID No.: Q9UBX7), ADAM-TS 15 (UniProt ID No.: Q8TE58), SYND1 (UniProt ID No.: P18827), CXCL13 (UniProt ID No.: 043927), TFPI-2 (UniProt ID No.: P48307), FR-α (UniProt ID No.: P15328), KLK13 (UniProt ID No.: Q9UKR3), MSLN (UniProt ID No.: Q13421), NECT4 (UniProt ID No.: Q96NY8), TNFRSF6B (UniProt ID No.: 095407), FCRLB (UniProt ID No.: Q6BAA4), and AREG (UniProt ID No.: P15514); and
identifying a patient with above normal levels of the one or more proteins as at risk for ovarian cancer.

16. A kit comprising:
a first reagent to measure a level of CA125 protein in a biological sample, the first reagent comprising an antibody and an oligonucleotide;
a second reagent to measure a level of HE4 protein in a biological sample, the second reagent comprising an antibody and an oligonucleotide;
a third reagent to measure a level of ITGAV protein in a biological sample, the third reagent comprising an antibody and an oligonucleotide;
a fourth reagent to measure a level of SEZ6L protein in a biological sample, the fourth reagent comprising an antibody and an oligonucleotide; and
a plate comprising wells, wherein a first well comprises the first reagent, a second well comprises the second reagent, a third well comprises the third reagent, and a fourth well comprises the fourth reagent.

17. The kit of claim 16, wherein each of the first, second, third, and fourth reagents comprises an antibody and an oligonucleotide.

18. A system for performing the method of claim 1.

19. A system for performing the method of claim 6.

20. A computer program comprising a non-transitory computer readable medium on which is provided program instructions for steps of
providing levels of CA125 protein HE4 protein, ITGAV protein, and SEZ6L protein from a biological sample from a patient;
providing levels of CA125 protein, HE4 protein, ITGAV protein, and SEZ6L protein from at least one reference sample;
determining, for each protein, a difference in the level of protein in the biological sample and the reference sample, thereby providing a normalized level of each protein;
weighting the normalized level of CA125 using a first coefficient a, wherein a is a positive value;
weighting the normalized level of HE4 using a second coefficient b, wherein b is a positive value;
weighting the normalized level of ITGAV using a third coefficient c, wherein c is a negative value;
weighting the normalized level of SEZ6L, using a fourth coefficient d, wherein d is a negative value;
determining an intercept value X; and
determining an ovarian cancer risk score for the patient, wherein the ovarian cancer risk score is calculated using Formula II:
$$Risk score \% = 100 * ( \frac{{e}^{X + a ( CA 125 ) + b ( HE 4 ) + c ( ITGAV ) + d ( SEZ 6 L )}}{1 + {e}^{X + a ( CA 125 ) + b ( HE 4 ) + c ( ITGAV ) + d ( SEZ 6 L )}} ) \qquad Formula II$$
wherein the ovarian cancer risk score indicates a percent chance the patient has ovarian cancer.
